(19) United States
(12) Patent Application Publication
(10) Pub. No.: US 2009/0227562 A1
(43) Pub. Date: Sep. 10, 2009

(54) Combination of a Selective Noradrenaline Reuptake Unhibitor and a PDEV Inhibitor

(75) Inventor: Bernadette Hughes, Kent (GB)

Correspondence Address: PHARMACIA CORPORATION;GLOBAL PATENT DEPARTMENT, POST OFFICE BOX 1027, ST. LOUIS, MO, 63006, US

(73) Assignee: Pfizer Inc., New York, NY (US)

(21) Appl. No.: 11/573,522

(22) PCT Filed: Jul. 8, 2005
(86) PCT No.: PCT/IB05/02457

§ 371 Date: Feb. 9, 2007

Related U.S. Application Data
(60) Provisional application No. 60/606,302, filed on Aug. 31, 2004.

(30) Foreign Application Priority Data
Aug. 10, 2004 (GB) 04107777.0

(51) Int. Cl. A61K 31/397; A61K 31/5375; A61K 31/496; A61K 31/4985; A61K 31/53; A61P 25/00

(52) U.S. Cl. 514/210.21; 514/239.2; 514/252.16; 514/250; 514/243

(57) ABSTRACT

The present invention relates to a combination of a selective noradrenaline reuptake inhibitor (NRI) and a phospho-diesterase type 5 (PDEV) inhibitor. Such a combination is particularly useful in the treatment of pain.

The present invention relates to a combination of a selective noradrenaline reuptake inhibitor (NRI) and a phosphodiesterase type 5 (PDEV) inhibitor and to pharmaceutical compositions comprising and to the uses of such a combination.

Noradrenaline (norpepinephrine) is an important monoamine neurotransmitter in the central nervous system. Normal levels of noradrenaline lead to drive and the capacity for reward. Abnormality in noradrenergic transmission, particularly a lower than normal level of noradrenaline, results in various types of mental, behavioural, and neurological disorders, particularly depression, characterised by a variety of symptoms including a lack of energy, motivation, and interest in life (see R. J. Baldessarini, “Drugs and the Treatment of Psychiatric Disorders: Depression and Mania” in Goodman and Gilman's The Pharmacological Basis of Therapeutics, McGraw-Hill, NY, pp. 432-439, 1996).

Noradrenaline travels from the terminal of a first neuron across a small gap called the synaptic cleft and binds to receptor molecules on the surface of a second neuron. This binding elicits intracellular changes that initiate or activate a response or change in the second (postsynaptic) neuron. Inactivation of the neurotransmitter occurs primarily by reuptake of the neurotransmitter by the first (presynaptic) neuron. Compounds which block this reuptake, known as noradrenaline reuptake inhibitors (NRIs), can therefore raise synaptic levels of noradrenaline and help to correct abnormalities in noradrenergic transmission. An example of such a noradrenaline reuptake inhibitor is reboxetine, (R,R/S,S)-(2-[(2-ethoxyphenoxy)(phenyl)methyl]morpholine). Reboxetine has been shown to be effective in the short-term (i.e., less than eight weeks) and long-term treatment of depression (see, for example, S. A. Montgomery, Reboxetine: Additional Benefits to the Depressed Patient, Psychopharmacol (Oxf) 11:4 Suppl., S9-15 (Abstract), 1997. The use of a particular enantiomer of reboxetine, (S,S)-reboxetine, in the treatment of chronic pain, peripheral neuropathy, incontinence (including stress incontinence, genuine stress incontinence, and mixed incontinence), fibromyalgia and other somatoform disorders, and migraine headaches is disclosed in WO-A-01/01973.

A PDEV inhibitor is a compound which inhibits the activity of the cyclic guanosine 3′,5′-monophosphate phosphodiesterase type five (cGMP PDEV) enzyme. An example is sildenafil (5-[2-ethoxy-5-(4-methyl-1-piperazinylsulphonyl)phenyl]-1-methyl-3-n-propyl-1,6-dihydro-7H-pyrazolo[4,3-d]pyrimidin-7-one, Viagra®), which was first described as a treatment for a number of cardiovascular disorders and has subsequently proved to be the first orally effective treatment for male erectile dysfunction (MED). The use of PDEV inhibitors in the treatment of neuropathy has been described in EP-A-1129706 and WO-A-01/26659. The analgesic effects of sildenafil have recently been described in Jain et al, Brain Research, 909, 170-178 (2001); Asomoza-Espinosa et al, Eur. J. Pharm., 418, 195-200 (2001); and Mixcoatl-Zecutal et al, Eur. J. Pharm., 400, 81-87 (2001).

There is an ongoing need to provide better treatments for pain that are, for example, more active at lower doses, active against a wider spectrum of pain conditions, less prone to produce side effects, faster acting and longer acting.

It has now been surprisingly found that combination therapy with a selective noradrenaline reuptake inhibitor (NRI) and a phosphodiesterase type 5 (PDEV) inhibitor offers significant benefits in the treatment of pain, especially as compared with the use of either agent alone. Such a combination results unexpectedly in a synergistic effect, producing greater efficacy than either class of agent used singly.

It is now thought that blocking the re-uptake of noradrenaline at pain synapses in the spinal cord may decrease the perception of pain by inhibiting the transmission of pain signals up the neuraxis. It is further thought that inhibition of PDEV may improve vascular perfusion of peripheral nerves, which become compromised as a result of pain-inducing conditions such as chronic diabetes. The unexpected synergy observed in the treatment of pain with a combination of a selective noradrenaline reuptake inhibitor (NRI) and a phosphodiesterase type 5 (PDEV) inhibitor may thus be the result of both reducing the input of pain signals into the central nervous system and simultaneously attenuating the transmission of such signals. This could explain why the combined benefit of the agents exceeds the sum of the benefits provided by each agent used separately.

The invention therefore provides a combination of a selective noradrenaline reuptake inhibitor (NRI) and a phosphodiesterase type 5 (PDEV) inhibitor.

Further, the invention provides a pharmaceutical composition comprising a selective noradrenaline reuptake inhibitor (NRI), a phosphodiesterase type 5 (PDEV) inhibitor and a pharmaceutically acceptable excipient, diluent or carrier.

Further, the invention provides a combination of a selective noradrenaline reuptake inhibitor (NRI) and a phosphodiesterase type 5 (PDEV) inhibitor for use as a medicament.

Further, the invention provides the use of a selective noradrenaline reuptake inhibitor (NRI) or a phosphodiesterase type 5 (PDEV) inhibitor in the manufacture of a medicament for simultaneous, sequential or separate administration of both agents in the treatment of pain.

Further, the invention provides a combination of a selective noradrenaline reuptake inhibitor (NRI) and a phosphodiesterase type 5 (PDEV) inhibitor for simultaneous, sequential or separate administration in the treatment of pain.

Further, the invention provides a method of treating pain comprising administering simultaneously, sequentially or separately, to a mammal in need of such treatment, an effective amount of a selective noradrenaline reuptake inhibitor (NRI) and a phosphodiesterase type 5 (PDEV) inhibitor.

Further, the invention provides a kit comprising a selective noradrenaline reuptake inhibitor (NRI), a phosphodiesterase type 5 (PDEV) inhibitor and means for containing said compounds.

Further, the invention provides a product containing a selective noradrenaline reuptake inhibitor (NRI) and a phosphodiesterase type 5 (PDEV) inhibitor as a combined preparation for simultaneous, separate or sequential use in the treatment of pain.

The combination provided by the present invention is useful in the treatment of pain, which is a preferred use. Physiological pain is an important protective mechanism designed to warn of danger from potentially injurious stimuli from the external environment. The system operates through a specific set of primary sensory neurones and is exclusively activated by noxious stimuli via peripheral transducing mechanisms (see Millan, 1999, Prog. Neurobiol., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Acute pain and chronic pain often involve the same pathways but driven by pathophysiological processes and as such ceasing to provide a protective mechanism and instead contributing to debilitating symptoms associated with a wide range of disease states. When a substantial injury occurs to body tissue, via disease or trauma, the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful, allowing for repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter, 2000, Science, 288, 1765-1768). Clinical pain is present when discomfort and abnormal sensitivity feature among the patient's symptoms. Patients tend to be quite heterogeneous and may present with various pain symptoms. There are a number of typical pain subtypes including: 1) spontaneous pain which may be dull, burning, or stabbing; 2) exaggerated pain responses to noxious stimuli (hyperalgesia); and 3) pain produced by normally innocuous stimuli (allodynia-Meyer et al., 1994, Textbook of Pain, 13-44). Although patients with back pain, arthritic pain, CNS trauma, or neuropathic pain may have similar symptoms, the underlying mechanisms are different and, therefore, may require different treatment strategies. Therefore pain can be divided into a number of different areas, because of differing pathophysiology, including nociceptive, inflammatory and neuropathic pain. It should be noted that some types of pain have multiple aetiologies and thus can be classified in more than one area, e.g. back pain and cancer pain have both nociceptive and neuropathic components.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain (which may be tumour related pain, e.g. bone pain, headache, facial pain and visceral pain, or pain associated with cancer therapy, e.g. postchemotherapy syndrome, chronic postsurgical pain syndrome and post radiation syndrome, or a cancer-related acute pain syndrome, e.g. caused by therapeutic interactions resulting from chemotherapy, immunotherapy, hormonal therapy and radiotherapy) and back pain (which may be due to herniated or ruptured intervertabral discs or abnormalities of the lumber facet joints, sacroiliac joints, paraspinal muscles or the posterior longitudinal ligament).

Neuropathic pain is defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395).

Most patients with osteoarthritis seek medical attention because of the associated pain. Arthritis has a significant impact on psychosocial and physical function and is known to be the leading cause of disability in later life. Another type of inflammatory pain is the pain associated with inflammatory bowel disease (IBD).

Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

Other types of pain include:
pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis;
heart and vascular pain, including pain caused by angina, myocardical infarction, mitral stenosis, pericarditis, Raynaud's phenomenon, scleredoma and skeletal muscle ischemia;
head pain, such as migraine (including migraine with aura and migraine without aura), cluster headache and tension-type headache; and
orofacial pain, including dental pain and temporomandibular myofascial pain.

The combination of the present invention is useful in the treatment of all kinds of pain, particularly neuropathic pain, most particularly post-herpetic neuralgia, painful diabetic neuropathy and chronic lower back pain.

The combination of the present invention is also useful in the treatment of conditions other than pain. In particular, the combination provided by the present invention is useful in the treatment of nervous system disorders such as addictive disorders (including those due to alcohol, nicotine, and other psychoactive substances) and withdrawal syndrome, adjustment disorders (including depressed mood, anxiety, mixed anxiety and depressed mood, disturbance of conduct, and mixed disturbance of conduct and mood), age-associated learning and mental disorders (including Alzheimer's disease), anorexia nervosa, apathy, attention-deficit (or other cognitive) disorders due to general medical conditions, attention-deficit hyperactivity disorder (ADHD), bipolar disorder, bulimia nervosa, chronic fatigue syndrome, chronic or acute stress, conduct disorder, cyclothymic disorder, depression (including adolescent depression and minor depression), dysthymic disorder, fibromyalgia and other somatoform disorders (including somatization disorder, conversion disorder, hypochondriasis, body dysmorphic disorder, undifferentiated somatoform disorder, and somatoform NOS), generalized anxiety disorder, incontinence (e.g. stress incontinence, genuine stress incontinence, urge incontinence and mixed incontinence), inhalation disorders, intoxication disorders (alcohol addiction), mania, obesity, obsessive compulsive disorders and related spectrum disorders, oppositional defiant disorder, panic disorder, post-traumatic stress disorder, premenstrual dysphoric disorder (i.e. premenstrual syndrome and late luteal phase dysphoric disorder), psychotic disorders (including schizophrenia, schizoaffective and schizophreniform disorders), seasonal affective disorder, sleep disorders (such as narcolepsy and enuresis), social phobia (including social anxiety disorder), specific developmental disorders, selective serotonin reuptake inhibition (SSRI) “poop out” syndrome (wherein a patient fails to maintain a satisfactory response to SSRI therapy after an initial period of satisfactory response), Parkinson's disease, cognition and memory disorders, nerve growth disorders, memory loss, hyperamnesia, concentration disorders, learning disorders, dementia and TIC disorders (e.g. Tourette's Disease).

The combination of the invention is also useful in the treatment of urinary incontinence, such as genuine stress incontinence (GSI), stress urinary incontinence (SUI) or urinary incontinence in the elderly; overactive bladder (OAB), including idiopathic detrusor instability, detrusor overactivity secondary to neurological diseases (e.g. Parkinson's disease, multiple sclerosis, spinal cord injury and stroke) and detrusor overactivity secondary to bladder outflow obstruction (e.g. benign prostatic hyperplasia (BPH), urethral stricture or stenosis); nocturnal enuresis; urinary incontinence due to a combination of the above conditions (e.g. stress incontinence associated with overactive bladder); and lower urinary tract symptoms, such as frequency and urgency. The term OAB is intended to encompass both OAB wet and OAB dry.

The combination of the present invention is also useful in the treatment of mammalian sexual dysfunctions such as male erectile dysfunction, impotence, female sexual dysfunction, clitoral dysfunction, female hypoactive sexual desire disorder, female sexual arousal disorder, female sexual pain disorder, female sexual orgasmic dysfunction, dyspareunia, priapism in patients with sickle cell disease, sexual dysfunction due to spinal cord injury and selective serotonin re-uptake inhibitor-induced sexual dysfunction (e.g. ejaculatory delay).

Sexual dysfunction (SD) is a significant clinical problem which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self-esteem and disrupts personal relationships thereby inducing personal distress. In the clinic, SD disorders have been divided into female sexual dysfunction (FSD) disorders and male sexual dysfunction (MSD) disorders (Melman et al., J. Urology, 1999, 161, 5-11).

FSD can be defined as the difficulty or inability of a woman to find satisfaction in sexual expression. FSD is a collective term for several diverse female sexual disorders (Leiblum, S. R. (1998). Definition and classification of female sexual disorders. Int. J. Impotence Res., 10, S104-S106; Berman, J. R., Berman, L. & Goldstein, I. (1999). Female sexual dysfunction: Incidence, pathophysiology, evaluations and treatment options. Urology, 54, 385-391). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998). Definition and classification of female sexual disorders, Int. J. Impotence Res., 10, S104-S106). Desire or libido is the drive for sexual expression. Its manifestations often include sexual thoughts either when in the company of an interested partner or when exposed to other erotic stimuli. Arousal is the vascular response to sexual stimulation, an important component of which is genital engorgement and includes increased vaginal lubrication, elongation of the vagina and increased genital sensation/sensitivity. Orgasm is the release of sexual tension that has culminated during arousal.

Hence, FSD occurs when a woman has an inadequate or unsatisfactory response in any of these phases, usually desire, arousal or orgasm. FSD categories include hypoactive sexual desire disorder, sexual arousal disorder, orgasmic disorders and sexual pain disorders. Although the combination of the present invention is also useful in improving the genital response to sexual stimulation (as in female sexual arousal disorder), in doing so it may also improve the associated pain, distress and discomfort associated with intercourse and so treat other female sexual disorders.

The combination of the present invention is also useful in treatment or prophylaxis of hypoactive sexual desire disorder, sexual arousal disorder, orgasmic disorder and sexual pain disorder, more preferably for the treatment or prophylaxis of sexual arousal disorder, orgasmic disorder, and sexual pain disorder, and most preferably in the treatment or prophylaxis of sexual arousal disorder. Hypoactive sexual desire disorder is present if a woman has no or little desire to be sexual, and has no or few sexual thoughts or fantasies. This type of FSD can be caused by low testosterone levels, due either to natural menopause or to surgical menopause. Other causes include illness, medications, fatigue, depression and anxiety.

Female sexual arousal disorder (FSAD) is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants eg SSRIs or antihypertensive agents. Sexual pain disorders (includes dyspareunia and vaginismus) is characterised by pain resulting from penetration and may be caused by medications which reduce lubrication, endometriosis, pelvic inflammatory disease, inflammatory bowel disease or urinary tract problems.

The prevalence of FSD is difficult to gauge because the term covers several types of problem, some of which are difficult to measure, and because the interest in treating FSD is relatively recent. Many women's sexual problems are associated either directly with the female ageing process or with chronic illnesses such as diabetes and hypertension.

Because FSD consists of several subtypes that express symptoms in separate phases of the sexual response cycle, there is not a single therapy. Current treatment of FSD focuses principally on psychological or relationship issues. Treatment of FSD is gradually evolving as more clinical and basic science studies are dedicated to the investigation of this medical problem. Female sexual complaints are not all psychological in pathophysiology, especially for those individuals who may have a component of vasculogenic dysfunction (eg FSAD) contributing to the overall female sexual complaint. There are at present no drugs licensed for the treatment of FSD. Empirical drug therapy includes oestrogen administration (topically or as hormone replacement therapy), androgens or mood-altering drugs such as buspirone or trazodone. These treatment options are often unsatisfactory due to low efficacy or unacceptable side effects.

The Diagnostic and Statistical Manual (DSM) IV of the American Psychiatric Association defines Female Sexual Arousal Disorder (FSAD) as being: “a persistent or recurrent inability to attain or to maintain until completion of the sexual activity adequate lubrication-swelling response of sexual excitement. The disturbance must cause marked distress or interpersonal difficulty.” The arousal response consists of vasocongestion in the pelvis, vaginal lubrication and expansion and swelling of the external genitalia. The disturbance causes marked distress and/or interpersonal difficulty.

FSAD is a highly prevalent sexual disorder affecting pre-, peri- and post menopausal (±HRT) women. It is associated with concomitant disorders such as depression, cardiovascular diseases, diabetes and UG disorders. The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

Male sexual dysfunction (MSD) is generally associated with either erectile dysfunction, also known as male erectile dysfunction (MED) and/or ejaculatory disorders such as premature ejaculation, anorgasmia (unable to achieve orgasm) or desire disorders such as hypoactive sexual desire disorder (lack of interest in sex).

PE is a relatively common sexual dysfunction in men. It has been defined in several different ways but the most widely accepted is the Diagnostic and Statistical Manual of Mental Disorders IV one which states: “PE is a lifelong persistent or recurrent ejaculation with minimal sexual stimulation before, upon or shortly after penetration and before the patient wishes it. The clinician must take into account factors that affect duration of the excitement phase, such as age, novelty of the sexual partner or stimulation, and frequency of sexual activity. The disturbance causes marked distress of interpersonal difficulty.”

The International Classification of Diseases 10 definition states: “There is an inability to delay ejaculation sufficiently to enjoy lovemaking, manifest as either of the following: (1) occurrence of ejaculation before or very soon after the beginning of intercourse (if a time limit is required: before or within 15 seconds of the beginning of intercourse); (2) ejaculation occurs in the absence of sufficient erection to make intercourse possible. The problem is not the result of prolonged abstinence from sexual activity”

Other definitions which have been used include classification on the following criteria: related to partner's orgasm; duration between penetration and ejaculation; and number of thrust and capacity for voluntary control.

Psychological factors may be involved in PE, with relationship problems, anxiety, depression, prior sexual failure all playing a role.

Ejaculation is dependent on the sympathetic and parasympathetic nervous systems. Efferent impulses via the sympathetic nervous system to the vas deferens and the epididymis produce smooth muscle contraction, moving sperm into the posterior urethra. Similar contractions of the seminal vesicles, prostatic glands and the bulbouretheral glands increase the volume and fluid content of semen. Expulsion of semen is mediated by efferent impulses originating from a population of lumber spinothalamic cells in the lumbosacral spinal cord (Coolen & Truitt, Science, 2002, 297, 1566) which pass via the parasympathetic nervous system and cause rhythmic contractions of the bulbocavernous, ischiocavernous and pelvic floor muscles. Cortical control of ejaculation is still under debate in humans. In the rat the medial pre-optic area and the paraventricular nucleus of the hypothalamus seem to be involved in ejaculation.

Ejaculation comprises two separate components—emission and ejaculation. Emission is the deposition of seminal fluid and sperm from the distal epididymis, vas deferens, seminal vesicles and prostrate into the prostatic urethra. Subsequent to this deposition is the forcible expulsion of the seminal contents from the urethral meatus. Ejaculation is distinct from orgasm, which is purely a cerebral event. Often the two processes are coincidental.

The combination of the present invention is also useful in the treatment of pre-eclampsia, polycystic ovary syndrome, intrauterine growth disorder, female infertility, dysmennorhea, micturition disorder, urine storage disorder, type 2 diabetes, type 1 diabetes, impaired glucose tolerance, insulin resistance, metabolic syndrome, diabetic complications (such as diabetic ulcers, diabetic foot ulcers, diabetic leg ulcers, diabetic neuropathy, peripheral diabetic neuropathy, diabetic nephropathy, diabetic retinopathy and hepatic insulin sensitising substance (HISS) dependant insulin resistance), premature labour, dysmenorrhea, benign prostatic hyperplasia (BPH), bladder outlet obstruction, lower urinary tract syndrome, intermittent claudication, angina (including stable, unstable and variant Prinzmetal angina), hypertension (including essential hypertension, pulmonary hypertension, secondary hypertension, isolated systolic hypertension, hypertension associated with diabetes, hypertension associated with atherosclerosis and renovascular hypertension), coronary artery disease, congestive heart failure, atherosclerosis, conditions of reduced blood vessel patency (e.g. post-percutaneous transluminal coronary angioplasty), peripheral vascular disease, stroke, nitrate induced tolerance, bronchitis, allergic asthma, chronic asthma, allergic rhinitis, hypoxic vasoconstriction, chronic obstructive pulmonary disease, bronchitis, cystic fibrosis, reversible pulmonary vasoconstriction, decreased pulmonary vascular resistance, partial and global respiratory failure, diseases characterised by disorders of gut motility (e.g. irritable bowel syndrome, IBS), gastroparesis (including diabetic gastroparesis), gastric emptying disorder, Barrett's oesophagus, anorectal disorders, dysperistalsis, spastic esophageal motor disorders (such as hypertensive LES), diffuse spasm, functional dyspepsia, gastropathy (such as diabetic gastropathy leading to nausea, vomiting, abdominal pain and early satiety), chronic hypoxia, pre-eclampsia, Kawasaki's syndrome, multiple sclerosis, acute respiratory failure, psoriasis (including psoriasis associated with renal syndrome), necrosis, scarring, chronic and acute skin wounds, fibrosis (including lung fibrosis, dermal and corneal scarring, fibrosis following infection, trauma, surgery or thermal injury, scieroderma and other connective tissue disorders, fibrosis of the heart, muscle fibrosis, kidney fibrosis, chronic dermal ulceration and lipdermatosclerosis), post-surgical and idiophatic adhesions, inflammatory conditions of the skin (including lichen and associated conditions), ageing and all ageing associated degenerative disorders (including ageing of the skin), liver fibrosis of any etiology (including viral and non-viral hepatitis and liver cirrhosis), chronic pancreatitis, chronic thyroiditis, calcinosis (of any origin), conditions whose pathogenesis is related to the deposition/remodelling of a connective matrix, acne, cancer (such as polyp cancer, prostate cancer, breast cancer, lung cancer, leukaemia, renal cancer, Crohn's disease, liver cancer, chronic lymphocytic cancer and neoplasm), cancer metastasis, baldness, alopecia, sepsis, osteoporosis, tinnitus, hearing loss, Hirschsprung's disease, Myasthaenia gravis, Eisenmenger's syndrome, nutcracker oesophagus, anal fissure, hemorrhoids, hypoxic vasoconstriction, CREST syndrome, systemic lupus erythematosis, rheumatoid diseases (such as rheumatoid arthritis), thrombosis, systemic sclerosis, thromboembolism, myocardial infarction, coronary insufficiency, ischaemic heart disease, platelet aggregation, blood pressure instability during haemodialysis, ischaemia/reperfusion injury and coronary cardiopathy.

The combination of the present invention is also useful in the stabilisation of blood pressure during haemodialysis and increasing birth weight.

The combination of the present invention is also useful in the treatment of diseases and conditions of the eye such as glaucoma, optic neuropathy, central retinal vein occlusion, elevated intra-ocular pressure, retinal artery occlusion, optic blood flow disorders, ocular neuropathies, macular degeneration (including age-related macular degeneration), optic nerve disease (e.g. normotensive excavatory optic neuropathy, ischaemic optic neuropathy, toxic optic neuropathy, traumatic optic neuropathy, idiopathic optic neuropathy, optic nerve drusen and benign intracranial hypertension), retinal disease (e.g. retinal neovascularisation, ischaemic haematologic/rheologic disorders and toxic maculopathy), choroidal disease (e.g. ischaemic disorder of the posterior choroid, degenerative subretinal neovascularisation, diabetic choroidal ischaemia, inflammatory subretinal neovasculisation, non-age related choroidal ischaemia, degenerative drusen of the macula, macular retinal pigment epithelial atrophy, retinal pigment epithelial detachment, degenerative subretinal neovasculisation, wet age related macular degeneration, macular edema, familial drusen, macular disorders related to hypertension, angioma, papillitis, neuroretinitis, pigmentary retinal degenerative disorders, macular edema without vascular leakage, retinitis pigmentosa, early stage macular hole, choroidal neovascularisation, branch retinal vein occlusion, intermediate uveitis and idiopathic retinal telagiectasis), low ocular blood flow, low visual function, elevated intra-ocular pressure and retinal or arterial occlusion.

In the context of the present invention, a ‘selective’ noradrenaline uptake inhibitor is a compound which is a more potent inhibitor of noradrenaline reuptake than serotonin reuptake, particularly a compound which is a more potent inhibitor of noradrenaline reuptake than serotonin and dopamine reuptake. Such selectivity may be determined by measuring the inhibition constant (or Kᵢ value) of a compound for the serotonin reuptake site and dividing it by the Kᵢ value of the compound for the noradrenaline reuptake site. A lower value of Kᵢ for noradrenaline reuptake indicates greater binding affinity to noradrenaline receptors. A higher serotonin (Kᵢ)/noradrenaline (Kᵢ) ratio indicates a greater selectivity for binding to the norepinephrine receptor. Preferably, such selectivity (serotonin (Kᵢ)/norepinephrine (Kᵢ)) is at least 10 fold, more preferably at least 100 fold, more preferably still at least 1000 fold, most preferably at least 5000 fold. Inhibition constants (Kᵢ values), typically reported in units of nanomolars (nM), can be calculated from the IC₅₀ values according to the method set forth in Y. C. Cheng and W. H. Prusoff, “Relationship Between the Inhibitory Constant (Kᵢ) and the Concentration of Inhibitor Which Causes 50% Inhibition (IC₅₀) of an Enzymatic Reaction,” Biochemical Pharmacology, vol. 22, pp. 3099-3108 (1973). Suitable experimental details are disclosed in WO-A-01/01973.

The terms “noradrenaline” and “norepinephrine” are synonymous and are both used in this specification. Similarly the terms “noradrenaline reuptake inhibitor” and “norepinephrine reuptake inhibitor” are synonymous, and the term “selective noradrenaline reuptake inhibitor” means the same as “selective norepinephrine reuptake inhibitor”.

A preferred selective noradrenaline uptake inhibitor is reboxetine, disclosed in GB-A-2014981. Reboxetine exhibits only marginal serotonin reuptake inhibition and no dopamine reuptake inhibition. Chemically, reboxetine has two chiral centers and, therefore, in theory, could exist as two enantiomeric pairs of diastereomers, the (R,R) and (S,S) enantiomeric pair and the (R,S) and (S,R) enantiomeric pair. However, the generic name reboxetine refers to the commercially available racemic mixture of the (R,R) and (S,S) enantiomers, in a 1:1 ratio. Reboxetine is sold commercially under the trade names of EDRONAX™, PROLIFT™, VESTRA™, and NOREBOX™.

A particularly preferred selective noradrenaline reuptake inhibitor is the (S,S) enantiomer of reboxetine, disclosed in GB-A-2167407. The (S,S)-enantiomer of reboxetine possesses greatly enhanced selectivity for the inhibition norepinephrine reuptake over the inhibition of serotonin reuptake, as disclosed in WO-A-01/01973. Accordingly, WO-A-01/01973 discloses a method of selectively inhibiting reuptake of norepinephrine, the method comprising the step of administering a therapeutically effective amount of a composition to an individual, the composition comprising a compound having a pharmacological selectivity of serotonin (Kᵢ)/norepinephrine (Kₗ) of at least about 5000.

Examples of suitable PDEV inhibitors for use in the combination of the present invention are: the pyrazolo[4,3-d]pyrimidin-7-ones disclosed in EP-A-0463756; the pyrazolo[4,3-d]pyrimidin-7-ones disclosed in EP-A-0526004; the pyrazolo[4,3-d]pyrimidin-7-ones disclosed in WO-A-93/06104; the isomeric pyrazolo[3,4-d]pyrimidin-4-ones disclosed in WO-A-93/07149; the quinazolin-4-ones disclosed in WO-A-93/12095; the pyrido[3,2-d]pyrimidin-4-ones disclosed in WO-A-94/05661; the purin-6-ones disclosed in WO-A-94/00453; the pyrazolo[4,3-d]pyrimidin-7-ones disclosed in WO-A-98/49166; the pyrazolo[4,3-d]pyrimidin-7 -ones disclosed in WO-A-54333; the pyrazolo[4,3-d]pyrimidin-4-ones disclosed in EP-A-0995751; the pyrazolo[4,3-d]pyrimidin-7-ones disclosed in WO-A-00/24745; the pyrazolo[4,3-d]pyrimidin-4-ones disclosed in EP-A-0995750; the hexahydropyrazino[2′,1′:6,1]pyrido[3,4-b]indole-1,4-diones disclosed in WO-A-95/19978; the imidazo[5,1-f][1,2,4]triazin-ones disclosed in EP-A-1092719 and WO-A-99/24433; and the bicyclic compounds disclosed in WO-A-93/07124; all of which are incorporated herein by reference.

Further examples of suitable PDEV inhibitors for use in the combination of the present invention include: the pyrazolo[4,3-d]pyrimidin-7-ones disclosed in WO-A-01/27112; the pyrazolo[4,3-d]pyrimidin-7-ones disclosed in WO-A-01/27113; the compounds disclosed in EP-A-1092718 and the compounds disclosed in EP-A-1092719; the tricyclic compounds disclosed in EP-A-1241170; the alkyl sulphone compounds disclosed in WO-A-02/074774; the compounds disclosed in WO-A-02/072586; the compounds disclosed in WO-A-02/079203; the compounds described in WO-A-01/87882; the compounds described in WO-A-00/56719, e.g. BMS-341400; the compounds described in WO-A-99/64004, e.g. BMS-263504; the compounds described in EP-A-1057829 (Jordanian Pharmaceutical Manufacturing and Medical Equipment Company); the compounds described in EP-A-722936; the compounds described in WO-A-93/07124; the compounds described in WO-A-98/06722; the compounds described in WO-A-98/06722; the compounds described in EP-A-579496, in particular ONO1505 (Ono); the compounds described in WO-A-97/03070, in particular OPC35564 (Otsuka); and the compounds described in WO-A-02/074312; all of which are incorporated herein by reference.

Yet further examples of suitable PDEV inhibitors for use in the combination of the present invention include the carboline derivatives described in WO-A-03/000691, WO-A-02/098875, WO-A-02/064591, WO-A-02/064590 and WO-A-01/08688; the pyrazino[1′,2′:1,6]pyrido[3,4-B]indole 1,4-dione derivatives described in WO-A-02/098877; the tetracyclic compounds described in WO-A-02/098428; the compounds described in WO-A-02/088123 and WO-A-02/00656; the condensed pyrazindione derivatives described in WO-A-02/38563 and WO-A-02/000657; the indole derivatives described in WO-A-02/36593; the condensed pyrindole derivatives described in WO-A-02/28865 and WO-A-02/28859; the hexahydropyrazino[1′,2′:1,6]-pyrido[3,4-B]indole-1,4-dione derivatives described in WO-A-02/28858 and WO-A-01/94345; the fused heterocyclic derivatives described in WO-A-02/10166; the cyclic GMP specific phosphodiesterase inhibitors described in WO-A-02/00658; the tetracyclic diketopiperazine compounds described in WO-A-01/94347; the compounds described in WO-A-02/98877; and the compounds described in use application WO-A-02/19213; all of which are incorporated herein by reference.

Yet further examples of suitable PDEV inhibitors for use in the combination of the present invention include the compounds described in WO-A-01/64192, DE-A-10104800, WO-A-02/59126, DE-A-10104095, WO-A-02/49651, DE-A-10063224, DE-A-10060338, DE-A-10058662, WO-A-02/00660, WO-A-2004/096810 and WO-A-2005/049616, all of which are incorporated herein by reference.

Still other PDEV inhibitors useful in conjunction with the present combination invention include: 4-bromo-5-(pyridylmethylamino)-6-[3-(4-chlorophenyl)-propoxy]-3(2H)pyridazinone; 1-[4-[(1,3-benzodioxol-5-ylmethyl)amino]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo-[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidine-carboxylic acid, monosodium salt; Pharmaprojects No. 4516 (Glaxo Wellcome); Pharmaprojects No. 5051 (Bayer); Pharmaprojects No. 5064 (Kyowa Hakko; see WO-A-96/26940); Pharmaprojects No. 5069 (Schering Plough); ER-118585, E-8010, E-4021 and E-4010 (Eisai); Bay-38-3045 & 38-9456 (Bayer); FR181074, FR229934 and FR226807 (Fujisawa); TA-1032, T-0156 and TA-1790 (Tanabe Seiyaku); EMD82639 and EMR6203 (Merck); LAS34179 and LAS35917 (Almirall); Sch-51866; BMS-223131 (Bristol Myers Squibb); NCX911 (Nicox); and ABT-724 and ABT-670 (Abbott).

Preferred PDEV inhibitors for the use in the present combination invention include:
(i) 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil, Viagra®) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);
(ii) 5-(2-ethoxy-5-morpholinoacetylphenyl)-1-methyl-3-n-propyl-1,6-dihydro-7H-pyrazolo[4,3-d]pyrimidin-7-one (see EP-A-0526004);
(iii) 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO-A-98/49166);
(iv) 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO-A-99/54333);
(v) (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO-A-99/54333);
(vi) 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO-A-01/27113, Example 8);
(vii) 5-[2-iso-butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO-A-01/27113, Example 15);
(viii) 5-[2-ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO-A-01/27113, Example 66);
(ix) 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one (see WO-A-01/27112, Example 124);
(x) 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one (see WO-A-01/27112, Example 132);
(xi) (6R,12aR)-2,3,6,7,12,12a-hexahydro-2-methyl-6-(3,4-methylene-dioxyphenyl)pyrazino[2′,1′:6,1]pyrido[3,4-b]indole-1,4-dione (tadalafil, IC-351, Cialis®), i.e. the compound of examples 78 and 95 of WO-A-95/19978, as well as the compound of examples 1, 3, 7 and 8;
(xii) 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, Levitra®) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of WO-A-99/24433;
(xiii) the pyrazolo[4,3-d]pyrimidin-4-ones disclosed in WO-A-00/27848, in particular N-[[3-(4,7-dihydro-1-methyl-7-oxo-3-propyl-1H-pyrazolo[4,3-d]-pyrimidin-5-yl)-4-propxyphenyl]sulfonyl]-1-methyl-2-pyrrolidine-propanamide [DA-8159 (Example 68 of WO-A-00/27848)];
(xiv) the compound of example 11 of WO-A-93/07124;
(xv) 4-(4-chlorobenzyl)amino-6,7,8-trimethoxyquinazoline;
(xvi) 7,8-dihydro-8-oxo-6-[2-propoxyphenyl]-1H-imidazo[4,5-g]quinazoline;
(xvii) 1-[3-[1-[(4-fluorophenyl)methyl]-7,8-dihydro-8-oxo-1H-imidazo[4,5-g]quinazolin-6-yl]-4-propoxyphenyl]carboxamide;
(xviii) 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one;
(xix) 1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}-4-ethylpiperazine;
(xx) N-[1-(2-ethoxyethyl)-5-(N-ethyl-N-methylamino)-7-(4-methylpyridin-2-ylamino)-1H-pyrazolo[4,3-d]pyrimidine-3-carbonyl]methanesulfonamide (Example 115 of WO-A-2005/049616);
(xxi) N-{5-(Ethyl-methyl-amino)-7-(4-methyl-pyridin-2-ylamino)-1-[2-(2,2,2-trifluoroethoxy)-ethyl]-1H-pyrazolo[4,3-d]pyrimidine-3-carbonyl}-methanesulfonamide;
(xxii) N-[5-(2,5-Diaza-bicyclo[2.2.1]hept-2-yl)-1-(2-ethoxyethyl)-3-ethyl-1H-pyrazolo[4,3-d]pyrimidin-7-yl]-(4-methylpyridin-2-yl)-amine, particularly N-[5-(1 S,4S)-(2,5-diaza-bicyclo[2.2.1]hept-2-yl)-1-(2-ethoxyethyl)-3-ethyl-1H-pyrazolo[4,3-d]pyrimidin-7-yl]-(4-methylpyridin-2-yl)-amine (Example 229 of WO-A-2004/098610); and
(xxiii) {3-Ethyl-5-[(R)-3-methyl-piperazin-1-yl]-1-[2-(2,2,2-trifluoroethoxy)-ethyl]-1H-pyrazolo[4,3-d]pyrimidin-7-yl}-pyrimidin-4-yl-amine (Example 249 of WO-A-2004/098610);
   and pharmaceutically acceptable salts and solvates thereof.

A further preferred PDEV inhibitor for use in the present combination invention is a compound of formula (III):

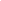

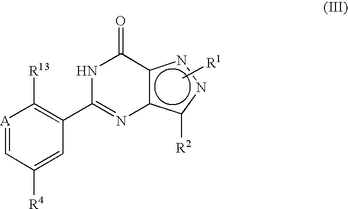

wherein:
A is CH or N;
R¹ is H, C₁ to C₆ alkyl, C₃ to C₆ alkenyl, C₃ to C₆ cycloalkyl, C₃ to C₆ cycloalkenyl, or C₁-C₃ perfluoroalkyl, wherein said alkyl group may be branched or straight chain and wherein said alkyl, alkenyl, cycloalkyl or perfluoroalkyl group is optionally substituted by; one or more substituents selected from: hydroxy; C₁ to C₄ alkoxy; C₃ to C₆ cycloalkyl; C₁-C₃ perfluoroalkyl; phenyl substituted with one or more substitutents selected from C₁ to C₃ alkyl, C₁ to C₄ alkoxy, C₁ to C₄ haloalkyl or C₁ to C₄ haloalkoxy wherein said haloalkyl and haloalkoxy groups contain one or more halo atoms, halo, CN, NO₂, NHR¹¹, NHSO₂R¹², SO₂R¹², SO₂NHR¹¹, COR¹¹, CO₂R¹¹ wherein R¹¹ is H, C₁ to C₄ alkyl, C₂ to C₄ alkenyl, C₁ to C₄ alkanoyl, C₁ to C₄ haloalkyl or C₁ to C₄ haloalkoxy and wherein R¹² is C₁ to C₄ alkyl, C₂ to C₄ alkenyl, C₁ to C₄ alkanoyl, C₁ to C₄ haloalkyl or C₁ to C₄ haloalkoxy; NR⁷R⁸, CONR⁷R⁸ or NR⁷COR¹¹ wherein R⁷ and R⁸ are each independently selected from H, C₁ to C₄ alkyl, C₂ to C₄ alkenyl, C₁ to C₄ alkoxy, CO₂R⁹, SO₂R⁹ wherein said alkyl, alkenyl or alkoxy groups are optionally substituted by NR⁵R⁶, C to C₄ haloalkyl or C₁ to C₄ haloalkoxy and wherein R⁹ is H, hydroxy C₂ to C₃ alkyl, C₁ to C₄ alkanoyl or C₁ to C₄ alkyl which is optionally substituted with phenyl wherein said phenyl group is optionally substituted by one or more substituents selected from C₁ to C₄ alkyl optionally substituted by C₁ to C₄ haloalkyl or C₁ to C₄ haloalkoxy, C₁ to C₄ alkoxy, halo, CN, NO₂, NHR¹¹, NHSO₂R¹², SO₂R¹², SO₂NHR¹¹, COR¹¹ or CO₂R¹¹; Het¹; Het² or Het³; or R¹ is Het⁴ or phenyl wherein said phenyl group is optionally substituted by one or more substituents selected from C₁ to C₄ alkyl, C₂ to C₄ alkenyl, C₁ to C₄ alkoxy, halo, CN, CF₃, OCF₃, NO₂, NHR¹¹, NHSO₂R¹², SO₂R¹², SO₂NHR¹¹, COR¹¹, CO₂R¹¹;
R² is H, C₁ to C₆ alkyl, C₃ to C₆ alkenyl or (CH₂)ₙ(C₃ to C₆ cycloalkyl) wherein n is 0, 1 or 2 and wherein said alkyl or alkyenyl group is optionally substituted with one or more fluoro substituents;
R¹³ is OR³ or NR⁵R⁶;
R³ is C₁ to C₆ alkyl, C₃-C₆ alkenyl, C₃-C₆ alkynyl, C₃-C₇ cycloalkyl, C₁-C₆ perfluoroalkyl or (C₃-C₆ cycloalkyl)C₁-C₆ alkyl optionally substituted with one or two substituents selected from C₃ to C₅ cycloalkyl, hydroxy, C₁ to C₄ alkoxy, C₃-C₆ alkenyl, C₃-C₆ alkynyl, benzyloxy, NR⁵R⁶, phenyl, Het¹, Het², Het³ or Het⁴ wherein the C₁ to C₆ alkyl and C₁ to C₄ alkoxy groups may optionally be terminated by a haloalkyl group such as CF₃; C₃ to C₆ cycloalkyl; Het¹, Het², Het³ or Het⁴;
R⁴ is C₁-C₄ alkyl optionally substituted with OH, NR⁵R⁶, CN, CONR⁵R⁶ or CO₂R⁷; C₂-C₄ alkenyl optionally substituted with CN, CONR⁵R⁶ or CO₂R⁷; C₂-C₄ alkanoyl optionally substituted with NR⁵R⁶; hydroxy C₂-C₄ alkyl optionally substituted with NR⁵R⁶; (C₂-C₃ alkoxy)C₁-C₂ alkyl optionally substituted with OH or NR⁵R⁵; CONR⁵R⁶; CO₂R⁷; halo; NR⁵R⁶; NHSO₂NR⁵R⁶; NHSO₂R⁸; or phenyl or heterocyclyl either of which is optionally substituted with methyl; or R⁴ is a pyrrolidinylsulphonyl, piperidinosulphonyl, morpholinosulphonyl, or piperazin-1-ylsulphonyl group having a substituent, R¹⁰ at the 4-position of the piperazinyl group wherein said piperazinyl group is optionally substituted with one or two C₁ to C₄ alkyl, C₁ to C₃ alkoxy, NR⁷R⁸ or CON R⁷R⁸ groups and is optionally in the form of its 4-N-oxide;
R⁵ and R⁶ are each independently selected from H and C₁ to C₄ alkyl optionally substituted with C₃ to C₅ cycloalkyl or C₁ to C₄ alkoxy, or, together with the nitrogen atom to which they are attached, form an azetidinyl, pyrrolidinyl, piperidinyl, morpholinyl, 4-(NR⁹)— piperazinyl or imidazolyl group wherein said group is optionally substituted with methyl or hydroxy;
R¹⁰ is H; C₁ to C₆ alkyl, (C₁-C₃ alkoxy) C₂-C₆ alkyl, hydroxy C₂-C₆ alkyl, (R⁷R⁸N)C₂-C₆ alkyl, (R⁷R⁸NCO)C₁-C₆ alkyl, CONR⁷R⁸, CSNR⁷R⁸ or C(NH)NR⁷R⁸ optionally substituted with one or two substituents selected from hydroxy, NR⁵R⁶, CONR⁵R⁶, phenyl optionally substituted with C₁ to C₄ alkyl or C₁ to C₄ alkoxy; C₂ to C₆ alkenyl or Het⁴;
Het¹ is an N-linked 4-, 5- or 6-membered nitrogen-containing heterocyclic group optionally containing one or more further heteroatoms selected from S, N or O;
Het² is a C-linked 5-membered heterocyclic group containing an O, S or N heteroatom optionally containing one or more heteroatoms selected from O or S;
Het³ is a C-linked 6-membered heterocyclic group containing an O or S heteroatom optionally containing one or more heteroatoms selected from O, S or N or Het³ is a C-linked 6-membered heterocyclic group containing three N heteroatoms;
Het⁴ is a C-linked 4-, 5- or 6-membered heterocyclic group containing one, two or three heteroatoms selected from S, O or N; and wherein any of said heterocyclic groups Het¹, Het², Het³ or Het⁴ may be saturated, partially unsaturated or aromatic and wherein any of said heterocyclic groups may be optionally substituted with one or more substituents selected from C₁ to C₄ alkyl, C₂ to C₄ alkenyl, C₁ to C₄ alkoxy, halo, CO₂R¹¹, COR¹¹, SO₂R¹² or NHR¹¹ and/or wherein any of said heterocyclic groups is benzo-fused;
or wherein when R¹³ represents OR³ or R³NR⁵; R¹ represents Het, alkylHet, aryl or alkylaryl, which latter five groups are all optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; R² represents H, halo, cyano, nitro, OR⁶, OC(O)R⁷, C(O)R⁸, C(O)OR⁹, C(O)NR¹⁰R¹¹, NR¹²R¹³, SO₂NR¹⁴R¹⁵, lower alkyl, Het, alkylHet, aryl or alkylaryl, which latter five groups are all optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; R³ represents H, lower alkyl, alkylHet or alkylaryl, which latter three groups are all optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; R⁴ represents H, halo, cyano, nitro, halo(loweralkyl), OR⁶, OC(O)R⁷, C(O)R⁸, C(O)OR⁹, C(O)NR¹⁰R¹¹, NR¹²R¹³, NR¹⁶Y(O)R¹⁷, SOR¹⁸, SO₂R¹⁹R²⁰, C(O)AZ, lower alkyl, lower alkenyl, lower alkynyl, Het, alkylHet, aryl, alkylaryl, which latter seven groups are all optionally substituted and/or terminated with one or more substituents selected from halo, cyano, nitro, lower alkyl, halo(loweralkyl), OR⁶OC(O)R⁷, C(O)R⁸, C(O)OR⁹, C(O)NR¹⁰R¹¹, NR¹²R¹³ and SO₂NR¹⁴R¹⁵; Y represents C or S(O), wherein one of R¹⁶ and R¹⁷ is not present when Y is S(O); A represents lower alkylene; Z represents OR⁶, halo, Het or aryl, which latter two groups are both optionally substituted with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵; R⁵, R⁶, R⁷, R⁸, R⁹, R¹⁸, R¹⁹ and R²⁰ independently represent H or lower alkyl; R¹⁰ and R¹¹ independently represent H or lower alkyl, which latter group is optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵ or Het or aryl optionally substituted with one or more of said latter eleven groups or one of R¹⁰ and R¹¹ may be lower alkoxy, amino or Het, which latter two groups are both optionally substituted with lower alkyl; R¹² and R¹³ independently represent H or lower alkyl or one of R¹² or R¹³ may be C(O)-lower alkyl or C(O)Het in which Het is optionally substituted with lower alkyl; R¹⁴ and R¹⁵ independently represent H or lower alkyl or R¹⁴ and R¹⁵, together with the nitrogen atom to which they are bound, form a heterocyclic ring; R¹⁶ and R¹⁷ independently represent H or lower alkyl or one of R¹⁶ and R¹⁷ may be Het or aryl, which latter two groups are both optionally substituted with: lower alkyl; Het represents an optionally substituted four to twelve membered heterocyclic group, which may be aromatic or non-aromatic, which may contain one or more double bonds, which may be mono- or bi-cyclic and which contains one or more heteroatoms selected from N, S and O;
   or a pharmaceutically acceptable salt or solvate of any thereof.

In formula (III), the PDEV inhibitor may contain halo groups. Here, “halo” means fluoro, chloro, bromo or iodo.

In formula (III), the PDE5 inhibitor may contain one or more of alkyl, alkoxy, alkenyl, alkylene and alkenylene groups—which may be unbranched—or branched-chain.

In formula (III), a preferred group of compounds for use according to the present invention are those wherein: R¹ is H, methyl or ethyl; R² is H, C₁-C₃ alkyl optionally substituted by OH, or methoxy; R³ is C₂-C₃ alkyl or allyl; R⁴ is a sulphonylpiperidino or 4-N—(R¹⁰)-sulphonylpiperazin-1-yl group; R⁵ is H, NR⁷R⁸, or CONR⁷R⁸; R¹⁰ is H, C₁-C₃ alkyl, hydroxy C₂-C₆ alkyl, CONR⁷R⁸, CSNR⁷R⁸ or C(NH)NR⁷R⁸; R⁷ and R⁸ are each independently H or methyl.

In formula (III), another preferred group of compounds for use according to the present invention are those wherein: R¹1 is C₁ to C₂ alkyl optionally substituted with Het; 2-(morpholin-4-yl)ethyl or benzyl; R² is C₂ to C₄ alkyl; R¹³ is OR³ or NR⁵R⁶; R³ is C₁ to C₄ alkyl optionally substituted with one or two substituents selected from cyclopropyl, cyclobutyl, OH, methoxy, ethoxy, benzyloxy, NR⁵R⁶, phenyl, furan-3-yl, pyridin-2-yl and pyridin-3-yl; cyclobutyl; 1-methylpiperidin-4-yl; tetrahydrofuran-3-yl or tetrahydropyran-4-yl; R⁵ and R⁶ are each independently selected from H and C₁ to C₂ alkyl optionally substituted with cyclopropyl or methoxy, or, together with the nitrogen atom to which they are attached, form a azetidinyl, pyrrolidinyl or morpholinyl group; R⁷ and R⁸, together with the nitrogen atom to which they are attached, form a 4—R¹⁰-piperazinyl group optionally substituted with one or two methyl groups and optionally in the form of its 4-N-oxide; R¹⁰ is H, C₁ to C₃ alkyl optionally substituted with one or two substituents selected from OH, NR⁵R⁶, CONR⁵R⁶, phenyl optionally substituted with methoxy, benzodioxol-5-yl and benzodioxan-2-yl; allyl; pyridin-2-yl; pyridin-4-yl or pyrimidin-2-yl; and Het is selected from pyridin-2-yl; 1-oxidopyridin-2-yl; 6-methylpyridin-2-yl; 6-methoxypyridin-2-yl; pyridazin-3-yl; pyrimidin-2-yl and 1-methylimidazol-2-yl. Of this group more preferred are those compounds wherein R¹ is C₁ to C₂ alkyl optionally substituted with Het; 2-(morpholin-4-yl)ethyl or benzyl; R² is C₂ to C₄ alkyl; R¹³ is OR³; R³ is C₁ to C₄ alkyl optionally monosubstituted with cyclopropyl, cyclobutyl, OH, methoxy, ethoxy, phenyl, furan-3-yl or pyridin-2-yl; cyclobutyl; tetrahydrofuran-3-yl or tetrahydropyran-4-yl; R⁷ and R⁸, together with the nitrogen atom to which they are attached, form a 4—R¹¹-piperazinyl group optionally in the form of its 4-N-oxide; R¹⁰ is C₁ to C₃ alkyl optionally monosubstituted with OH; and Het is selected from pyridin-2-yl; 1-oxidopyridin-2-yl; 6-methylpyridin-2-yl; 6-methoxypyridin-2-yl; pyridazin-3-yl; pyrimidin-2-yl and 1-methylimidazol-2-yl.

In formula (III), one other further preferred group of compounds for use according to the present invention are those wherein: R¹ is C₁ to C₆ alkyl or C₃ to C₆ alkenyl wherein said alkyl or alkenyl groups may be branched chain or straight chain or R¹ is C₃ to C₆ cycloalkyl or C₄ to C₆ cycloalkenyl and wherein when R¹ is C₁ to C₃ alkyl said alkyl group is substituted by; and wherein when R¹ is C₄ to C₆ alkyl, C₃ to C₆ alkenyl, C₃ to C₆ cycloalkyl or C₄ to C₆ cycloalkenyl said alkyl, alkenyl, cycloalkyl or cycloalkenyl group is optionally substituted by; one or more substituents selected from: hydroxy; C₁ to C₄ alkoxy; C₃ to C₄ cycloalkyl; phenyl substituted with one or more substitutents selected from C₁ to C₃ alkyl, C₁ to C₄ alkoxy, C₁ to C₄ haloalkyl or C₁ to C₄ haloalkoxy, halo, CN, NO₂, NHR¹¹, NHCOR¹², NHSO₂R¹², SO₂R¹², SO₂NHR¹¹, COR¹¹, CO₂R¹¹ wherein said haloalkyl and haloalkoxy groups contain one or more halo atoms; NR⁷R⁸, CONR⁷R⁸ or NR⁷COR¹¹; a Het¹ group which is an N-linked 4-membered N-containing heterocyclic group; a Het² group which is a C-linked 5-membered heterocyclic group containing an O, S or N heteroatom optionally containing one or more heteroatoms selected from N, O or S; a Het³ group which is a C-linked 6-membered heterocyclic group containing an O or S heteroatom optionally containing one or more heteroatoms selected from O, S or N or a Het³ group which is a C-linked 6-membered heterocyclic group containing three N heteroatoms; wherein R⁷, R⁸, R¹¹ and R¹² are as previously defined herein or R¹ is a Het⁴. group which is a C-linked 4- or 5-membered heterocyclic group containing one heteroatom selected from S, O or N; a Het⁴ group which is a C-linked 6-membered heterocyclic group containing one, two or three heteroatoms selected from S or O; a Het⁴ group which is a C-linked 6-membered heterocyclic group containing three nitrogen heteroatoms; a Het⁴ group which is a C-linked 6-membered heterocyclic group containing one or two nitrogen heteroatoms which is substituted by one or more substitutents selected from C₁ to C₄ alkyl, C₁ to C₄ alkoxy, CO₂R¹¹, SO₂R¹², COR¹¹, NHR¹¹ or NHCOR¹² and optionally including a further heteroatom selected from S, O or N wherein any of said heterocyclic groups Het¹, Het², Het³ or Het⁴ is saturated, partially unsaturated or aromatic as appropriate and wherein any of said heterocyclic groups is optionally substituted with one or more substituents selected from C₁ to C₄ alkyl, C₃ to C₄ alkenyl, C₁ to C₄ alkoxy, halo, CO₂R¹¹, SO₂R¹², COR¹¹ or NHR¹¹ wherein R¹¹ is as defined hereinbefore and/or wherein any of said heterocyclic groups is benzo-fused; or R¹ is phenyl substituted by one or more substituents selected from CF₃, OCF₃, SO₂R¹² or CO₂R¹² wherein R¹² is C₁ to C₄ alkyl which is optionally substituted by phenyl, C₁ to C₄ haloalkyl or C₁ to C₄ haloalkoxy wherein said haloalkyl and haloalkoxy groups contain one or more halo atoms; R² is C₁ to C₅ alkyl; R¹³ is OR³; R³ is C₁ to C₆ alkyl optionally substituted with one or two substituents selected from C₃ to C₅ cycloalkyl, hydroxy, C₁ to C₄ alkoxy, benzyloxy, NR⁵R⁶, phenyl, furanyl, tetrahydrofuranyl or pyridinyl wherein said C₁ to C₆ alkyl and C₁ to C₄ alkoxy groups may optionally be terminated by a haloalkyl group such as CF₃; or R³ is C₃ to C₆ cycloalkyl, 1-(C₁ to C₄ alkyl)piperidinyl, tetrahydrofuranyl or tetrahydropyranyl; R⁴ is a piperazin-1-ylsulphonyl group having a substituent R¹⁰ at the 4-position of the piperazinyl group wherein said piperazinyl group is optionally substituted with one or two C₁ to C₄ alkyl groups and is optionally in the form of its 4-N-oxide; R⁵ and R⁶ are each independently selected from H and C₁ to C₄ alkyl optionally substituted with C₃ to C₅ cycloalkyl or C₁ to C₄ alkoxy, or, together with the nitrogen atom to which they are attached, form an azetidinyl, pyrrolidinyl, piperidinyl or morpholinyl group; and R¹⁰ is H; C₁ to C₄ alkyl optionally substituted with one or two substituents selected from hydroxy, NR⁵R⁶, CONR⁵R⁶, phenyl optionally substituted with C₁ to C₄ alkyl or C₁ to C₄ alkoxy; C₃ to C₆ alkenyl; Het⁴; with the proviso that when R¹ is C₁ to C₃ alkyl substituted by phenyl then said phenyl group is not substituted by C₁ to C₄ alkoxy; CN; halo; CF₃; OCF₃; or C₁ to C₄ alkyl. More preferred of this group of compounds are those wherein R¹ is C₁ to C₆ alkyl wherein said alkyl may be branched or straight chain or R¹ is C₃ to C₆ cycloalkyl and wherein when R¹ is C₁ to C₃ alkyl said alkyl group is substituted by; and wherein when R¹ is C₄ to C₆ alkyl or C₃ to C₆ cycloalkyl said alkyl or cycloalkyl group is optionally substituted by; one or more substituents selected from: hydroxy; C₁ to C₂ alkoxy; C₃ to C₅ cycloalkyl; NR⁷R⁸, NR⁷COR¹¹ or COR¹¹ wherein R⁷ and R⁸ are each independently selected from H, C₁ to C₄ alkyl or CO₂R⁹ wherein R⁹ and R¹¹ are as previously defined herein; a Het¹ group which is an N-linked 4-membered N-containing heterocyclic group; a Het³ group which is a C-linked 6-membered heterocyclic group containing an 0 or S heteroatom optionally containing one or more heteroatoms selected from O, S or N or a Het³ group which is a C-linked 6-membered heterocyclic group containing three N heteroatoms; or R¹ is a Het⁴ group which is a C-linked 4-membered heterocyclic group containing one heteroatom selected from S, O or N or R¹ is a Het⁴ group which is a C-linked 6-membered heterocyclic group containing one, two or three heteroatoms selected from S or O wherein any of said heterocyclic groups Het¹, Het², Het³ or Het⁴ is saturated, partially unsaturated or aromatic and is optionally substituted with one or more substituents selected from C₁ to C₄ alkyl, C₁ to C₄ alkoxy, —CO₂R¹¹, —SO₂R¹², —COR¹¹ or NHR¹¹ wherein R¹¹ and R¹² are as defined hereinbefore and/or wherein any of said heterocyclic groups is benzo-fused; or R¹ is phenyl substituted by one or more substituents selected from: CF₃, —OCF₃, —SO₂R¹², —COR¹¹, —CO₂R¹¹ wherein R¹¹ and R¹² are as defined hereinbefore; R² is C₁ to C₆ alkyl; R¹³ is OR³; R³ is methyl, ethyl, n-propyl, i-propyl, n-butyl, sec-butyl, 1-butyl or t-butyl alkyl optionally substituted with one or two substituents selected from cyclopropyl, cyclobutyl, hydroxy, methoxy, ethoxy, benzyloxy, phenyl, benzyl, furan-3-yl, tetrahydrofuran-2-ylmethyl, tetrahydrofuran-3-ylmethyl, pyridin-2-yl, pyridin-3-yl or NR⁵R⁶ wherein R⁵ and R⁶ are each independently selected from H and C₁ to C₂ alkyl; R⁴ is a piperazin-1-ylsulphonyl group having a substituent, R¹⁰ at the 4-position of the piperazinyl group wherein said piperazinyl group is optionally substituted with one or two C₁ to C₄ alkyl groups and is optionally in the form of its 4-N-oxide; and R¹⁰ is H, C₁ to C₃ alkyl optionally substituted with one or two substituents selected from hydroxy, NR⁵R⁶, CONR⁵R⁶ wherein R⁵ and R⁶ are each independently selected from H, C₁ to C₄ alkyl and C₃ alkenyl.

In formula (III), a further group of preferred compounds for use according to the present invention are those wherein: R¹ represents H, lower alkyl, Het, alkylHet, or alkylaryl (which latter four groups are all optionally substituted and/or terminated with one or more substituents selected from cyano, lower alkyl, OR⁶, C(O)OR⁹ or NR¹²R¹³); R² represents H, halo, lower alkyl, Het or aryl (which latter three groups are all optionally substituted and/or terminated with one or more substituents as defined hereinbefore, and preferably with NR¹²R¹³ or SO₂NR¹⁴R¹⁵); R³ represents C₁-C₄ alkyl or C₃-C₄ cycloalkyl which are optionally substituted and/or terminated with one or more substituents selected from halo, cyano, nitro, lower alkyl, halo(loweralkyl), OR⁶, OC(O)R⁷, C(O)R⁸, C(O)OR⁹, C(O)NR¹⁰R¹¹, NR¹²R¹³ and SO₂NR¹⁴R¹⁵); R⁴ represents halo, cyano, nitro, C(O)R⁸, C(O)OR⁹, C(O)NR¹⁰R¹¹, NR¹²R¹³, N[Y(O)R¹⁷]₂, NR¹⁶Y(O)R¹⁷, SOR¹⁸, SO₂R¹⁹, C(O)AZ, lower alkyl, lower alkynyl, Het or aryl, which latter three groups are all optionally substituted and/or terminated with one or more substituents as defined hereinbefore; and wherein Y, A, Z, R¹⁰, R¹¹, R¹², R¹³, R⁴, R¹⁵, R¹⁶, R¹⁷, R⁵, R⁶, R⁷, R⁸, R⁹, R¹⁸, R¹⁹ and Het are as herein before defined. More preferred in this further group are compounds in which R¹ represents optionally substituted lower alkyl, more preferably lower alkyl, lower alkoxy-terminated lower alkyl, NR¹²R¹³-terminated lower alkyl, or N-morpholino-erminated lower alkyl. Alternatively, R¹ may represent a 4-piperidinyl or a 3-azetidinyl group, optionally substituted at the nitrogen atom of the piperidinyl group with lower alkyl or C(O)OR⁹. In such more preferred compounds in this further group R² represents C(O)NR¹⁰R¹¹, NR¹²R¹³, lower alkyl optionally interrupted by one or more of O, S or N, optionally substituted at N by lower alkyl or acyl, or optionally substituted aryl or Het. More preferably, when R² is interrupted lower alkyl, the interrupting atoms are one or more of O and lower alkylated-N and when R² is aryl, it is optionally substituted phenyl or pyridyl. Particularly preferred compounds of this further group are those in which R² represents C(O)NR¹⁰R¹¹, NR¹²R¹³, C₁₋₄ alkyl optionally interrupted by O or N, optionally substituted at N by lower alkyl, optionally substituted phenyl, or optionally substituted pyridin-2-yl, pyridin-3-yl, pyrimidin-5-yl, pyrazin-2-yl, pyrazol-4-yl, oxadiazol-2-yl, furan-2-yl, furan-3-yl, tetrahydrofuran-2-yl and imidazo[1,2-a]pyridin-6-yl. In this more preferred group of further compounds R³ may represent lower alkyl or cycloalkyl. Also, X is preferably O, Such further and more preferred compounds have R⁴ representing halo, lower alkyl, lower alkynyl, optionally substituted Het, optionally substituted aryl, C(O)R⁸, C(O)AZ, C(O)OR⁹, C(O)NR¹⁰R¹¹, NR¹²R¹³ or NR¹⁶Y(O)R¹⁷. More preferred values for R⁴ are C(O)R⁸ (e.g. acetyl), halo (e.g. iodo), SO₂R¹⁹ (wherein R¹⁹ represents lower alkyl) and C(O)NR¹⁰R¹¹ (e.g. where R¹⁰ and R¹¹ independently represent H and lower alkyl and/or one of R¹⁰ and R¹¹ is lower alkoxy) or NHB, wherein B represents H, SO₂CH₃ or C(O)Het. Further preferred still are compounds in which R⁴ represents iodo, lower alkyl, lower alkynyl (which latter two groups are substituted and/or terminated by C(O)OR⁹ (wherein R⁹ represents H or C₁₋₆ alkyl)), N(H)Y(O)R¹⁷, N[Y(O)R¹⁷]₂, optionally substituted Het or NR¹²R¹³ (wherein R¹² and R¹³ together represent C₃₋₅ alkylene interrupted by O or N—S(O)₂-(optionally substituted aryl)).

A preferred PDEV inhibitor for use in the present combination invention has an IC₅₀ for the PDEV enzyme of less than 100 nanomolar, more preferably, less than 50 nanomolar, more preferably still less than 10 nanomolar. Such IC₅₀ values may be determined using known PDE5 assays described, for example, in WO-A-01/27113, EP-A-0526004 and EP-B-0463756.

Preferably, a PDEV inhibitor used in accordance with the invention is selective for the PDEV enzyme (as measured in terms of IC₅₀). Preferably it has a selectivity for PDEV over PDE3 of greater than 100, more preferably greater than 300. More preferably the PDEV inhibitor has a selectivity over both PDE3 and PDE4 of greater than 100, more preferably greater than 300. Even more preferably, it has a selectivity over all other PDE enzymes of greater than 10, more preferably greater than 100. Selectivity ratios may readily be determined by the skilled person. IC50 values for the PDE3 and PDE4 enzyme may be determined using established literature methodology, see S A Ballard et al, Journal of Urology, 1998, vol. 159, pages 2164-2171.

Particularly preferred PDEV inhibitors for use in the present combination invention are:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) or a pharmaceutically acceptable salt or solvate thereof, particularly sildenafil citrate;
(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (tadalafil, IC-351, Cialis®) or a pharmaceutically acceptable salt or solvate thereof;
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, Levitra®) or a pharmaceutically acceptable salt or solvate thereof;
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one or a pharmaceutically acceptable salt or solvate thereof;
5-(5-acetyl-2-butoxy-3-pyridinyl)-3-ethyl-2-(1-ethyl-3-azetidinyl)-2,6-dihydro-7H-pyrazolo[4,3-d]pyrimidin-7-one or a pharmaceutically acceptable salt or solvate thereof;
1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}-4-ethylpiperazine or a pharmaceutically acceptable salt or solvate thereof;
N-[1-(2-ethoxyethyl)-5-(N-ethyl-N-methylamino)-7-(4-methylpyridin-2-ylamino)-1H-pyrazolo[4,3-d]pyrimidine-3-carbonyl]methanesulfonamide or a pharmaceutically acceptable salt or solvate thereof;
3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one or a pharmaceutically acceptable salt or solvate thereof; and
{3-ethyl-5-[(R)-3-methyl-piperazin-1-yl]-1-[2-(2,2,2-trifluoroethoxy)-ethyl]-1H-pyrazolo[4,3-d]pyrimidin-7-yl}pyrimidin-4-yl-amine or a pharmaceutically acceptable salt or solvate thereof.

A preferred combination according to the invention is the combination of a selective noradrenaline reuptake inhibitor (NRI) selected from:

reboxetine, or a pharmaceutically acceptable salt or solvate thereof, particularly reboxetine mesylate; and
(S,S)-reboxetine or a pharmaceutically acceptable salt or solvate thereof, particularly (S,S)-reboxetine succinate;
and a phosphodiesterase type 5 (PDEV) inhibitor selected from:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) or a pharmaceutically acceptable salt or solvate thereof, particularly sildenafil citrate;
(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (tadalafil, IC-351, Cialis®)) or a pharmaceutically acceptable salt or solvate thereof;
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) or a pharmaceutically acceptable salt or solvate thereof;
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one or a pharmaceutically acceptable salt or solvate thereof;
5-(5-acetyl-2-butoxy-3-pyridinyl)-3-ethyl-2-(1-ethyl-3-azetidinyl)-2,6-dihydro-7H-pyrazolo[4,3-d]pyrimidin-7-one or a pharmaceutically acceptable salt or solvate thereof;
1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}-4-ethylpiperazine or a pharmaceutically acceptable salt or solvate thereof;
N-[1-(2-ethoxyethyl)-5-(N-ethyl-N-methylamino)-7-(4-methylpyridin-2-ylamino)-1H-pyrazolo[4,3-d]pyrimidine-3-carbonyl]methanesulfonamide or a pharmaceutically acceptable salt or solvate thereof;
3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one or a pharmaceutically acceptable salt or solvate thereof; and {3-ethyl-5-[(R)-3-methyl-piperazin-1-yl]-1-[2-(2,2,2-trifluoroethoxy)-ethyl]-1H-pyrazolo[4,3-d]pyrimidin-7-yl}-pyrimidin-4-yl-amine or a pharmaceutically acceptable salt or solvate thereof.

A preferred combination according to the invention is the combination of (S,S)-reboxetine or a pharmaceutically acceptable salt or solvate thereof, particularly (S,S)-reboxetine succinate, and a phosphodiesterase type 5 (PDEV) inhibitor selected from:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) or a pharmaceutically acceptable salt or solvate thereof, particularly sildenafil citrate;
(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (tadalafil, IC-351, Cialis®) or a pharmaceutically acceptable salt or solvate thereof;
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, Levitra®) or a pharmaceutically acceptable salt or solvate thereof;
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one or a pharmaceutically acceptable salt or solvate thereof;
5-(5-acetyl-2-butoxy-3-pyridinyl)-3-ethyl-2-(1-ethyl-3-azetidinyl)-2,6-dihydro-7H-pyrazolo[4,3-d]pyrimidin-7-one or a pharmaceutically acceptable salt or solvate thereof;
1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}-4-ethylpiperazine or a pharmaceutically acceptable salt or solvate thereof;
N-[1-(2-ethoxyethyl)-5-(N-ethyl-N-methylamino)-7-(4-methylpyridin-2-ylamino)-1H-pyrazolo[4,3-d]pyrimidine-3-carbonyl]methanesulfonamide or a pharmaceutically acceptable salt or solvate thereof;
3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one or a pharmaceutically acceptable salt or solvate thereof; and
{3-ethyl-5-[(R)-3-methyl-piperazin-1-yl]-1-[2-(2,2,2-trifluoroethoxy)-ethyl]-1H-pyrazolo[4,3-d]pyrimidin-7-yl}-pyrimidin-4-yl-amine or a pharmaceutically acceptable salt or solvate thereof.

A preferred combination according to the invention is the combination of (S,S)-reboxetine or a pharmaceutically acceptable salt or solvate thereof, particularly (S,S)-reboxetine succinate, and 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) or a pharmaceutically acceptable salt or solvate thereof, particularly sildenafil citrate.

A preferred combination according to the invention is the combination of (S,S)-reboxetine or a pharmaceutically acceptable salt or solvate thereof, particularly (S,S)-reboxetine succinate, and (6R,12aR)-2,3,6,7,12,12a-hexahydro-2-methyl-6-(3,4-methylenedioxyphenyl)-pyrazino[2′, 1′:6,1]pyrido[3,4-b]indole-1,4-dione (tadalafil, IC-351, Cialis®) or a pharmaceutically acceptable salt or solvate thereof.

A preferred combination according to the invention is the combination of (S,S)-reboxetine or a pharmaceutically acceptable salt or solvate thereof, particularly (S,S)-reboxetine succinate, and 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) or a pharmaceutically acceptable salt or solvate thereof.

A preferred combination according to the invention is the combination of (S,S)-reboxetine or a pharmaceutically acceptable salt or solvate thereof, particularly (S,S)-reboxetine succinate, and 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one or a pharmaceutically acceptable salt or solvate thereof.

A preferred combination according to the invention is the combination of (S,S)-reboxetine or a pharmaceutically acceptable salt or solvate thereof, particularly (S,S)-reboxetine succinate, and 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one or a pharmaceutically acceptable salt or solvate thereof.

A preferred combination according to the invention is the combination of (S,S)-reboxetine or a pharmaceutically acceptable salt or solvate thereof, particularly (S,S)-reboxetine succinate, and 1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}-4-ethylpiperazine or a pharmaceutically acceptable salt or solvate thereof.

A preferred combination according to the invention is the combination of (S,S)-reboxetine or a pharmaceutically acceptable salt or solvate thereof, particularly (S,S)-reboxetine succinate, and N-[1-(2-ethoxyethyl)-5-(N-ethyl-N-methylamino)-7-(4-methylpyridin-2-ylamino)-1H-pyrazolo[4,3-d]pyrimidine-3-carbonyl]methanesulfonamide or a pharmaceutically acceptable salt or solvate thereof.

A preferred combination according to the invention is the combination of (S,S)-reboxetine or a pharmaceutically acceptable salt or solvate thereof, particularly (S,S)-reboxetine succinate, and 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one or a pharmaceutically acceptable salt or solvate thereof.

A preferred combination according to the invention is the combination of (S,S)-reboxetine or a pharmaceutically acceptable salt or solvate thereof, particularly (S,S)-reboxetine succinate, and {3-ethyl-5-[(R)-3-methyl-piperazin-1-yl]-1-[2-(2,2,2-trifluoroethoxy)-ethyl]-1H-pyrazolo[4,3-d]pyrimidin-7-yl}-pyrimidin-4-yl-amine or a pharmaceutically acceptable salt or solvate thereof.

A selective noradrenaline reuptake inhibitor (NRI) or a phosphodiesterase type 5 (PDEV) inhibitor selected for use in the combination of the present invention, particularly one of the suitable or preferred compounds listed above, (hereinafter referred to as ‘a compound for use in the invention’) may be used in the form of a pharmaceutically acceptable salt, for example an acid addition or base salt.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, Weinheim, Germany, 2002).

Pharmaceutically acceptable salts of a compound for use in the invention may be prepared by one or more of three methods:

(i) by reacting the compound with the desired acid or base;
(ii) by removing an acid- or base-labile protecting group from a suitable precursor of the compound or by ring-opening a suitable cyclic precursor, for example, a lactone or lactam, using the desired acid or base; or
(iii) by converting one salt of the compound to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

A compound for use in the invention may exist in both unsolvated and solvated forms. The term ‘solvate’ is used herein to describe a molecular complex comprising the compound and a stoichiometric amount of one or more pharmaceutically acceptable solvent molecules, for example, ethanol. The term ‘hydrate’ is employed when said solvent is water.

A compound for use in the invention may form a complex such as a clathrate, a drug-host inclusion complexe wherein, in contrast to the aforementioned solvates, the drug and host are present in stoichiometric or non-stoichiometric amounts. A compound for use in the invention may also contain two or more organic and/or inorganic components which may be in stoichiometric or non-stoichiometric amounts. The resulting complexes may be ionised, partially ionised, or non-ionised. For a review of such complexes, see J. Pharm. Sci., 64 (8), 1269-1288, by Haleblian (August 1975).

A compound for use in the invention may be used in the form of a pro-drug. Thus, certain derivatives of a compound which may have little or no pharmacological activity themselves can, when administered into or onto the body, be converted into compounds having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association). Prodrugs can, for example, be produced by replacing appropriate functionalities with certain moieties known to those skilled in the art as ‘pro-moieties’ as described, for example, in Design of Prodrugs by H. Bundgaard (Elsevier, 1985).

A compound for use in the invention may also form active metabolites when administered to a patient, mainly by oxidative processes. Hydroxylation by liver enzymes is of particular note.

A compound for use in the invention which contains one or more asymmetric carbon atoms can exist as two or more stereoisomers. Where a compound contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds containing, for example, an imino, keto, or oxime group, or so-called valence tautomerism in compounds which contain an aromatic moiety. It follows that a single compound may exhibit more than one type of isomerism.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Stereoisomeric conglomerates may be separated by conventional techniques known to those skilled in the art—see, for example, Stereochemistry of Organic Compounds by E. L. Eliel and S. H. Wilen (Wiley, New York, 1994).

A compound for use in the invention may be isotopically-labelled wherein one or more atoms are replaced by atoms having the same atomic number, but an atomic mass or mass number different from the atomic mass or mass number which predominates in nature.

Examples of such isotopes include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Pharmaceutically acceptable solvates include those wherein the solvent of crystallization may be isotopically substituted, e.g. D₂O, d₆-acetone, d₆-DMSO.

A compound for use in the invention may be administered as a crystalline or amorphous product. It may be obtained, for example, as a solid plug, powder or film by methods such as precipitation, crystallization, freeze drying, spray drying, or evaporative drying. Microwave or radio frequency drying may be used for this purpose.

A compound for use in the invention may be administered alone but will more likely be administered as a formulation in association with one or more pharmaceutically acceptable excipients. The term ‘excipient’ is used herein to describe any ingredient other than a compound for use in the invention. The choice of excipient will to a large extent depend on factors such as the particular mode of administration, the effect of the excipient on solubility and stability, and the nature of the dosage form.

Pharmaceutical compositions suitable for the delivery of a compound for use in the invention and methods for their preparation will be readily apparent to those skilled in the art. Such compositions and methods for their preparation may be found, for example, in Remington's Pharmaceutical Sciences, 19th Edition (Mack Publishing Company, 1995).

A compound for use in the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

A compound for use in the invention may also be used in a fast-dissolving, fast-disintegrating dosage form such as one of those described in Expert Opinion in Therapeutic Patents, 11 (6), 981-986, by Liang and Chen (2001).

For tablet dosage forms, depending on dose, a compound for use in the invention will generally make up from 1 weight % to 80 weight % of the dosage form, more typically from 5 weight % to 60 weight % of the dosage form. In addition, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight. % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound for use in the invention, a film-forming polymer, a binder, a solvent, a humectant, a plasticiser, a stabiliser or emulsifier, a viscosity-modifying agent and a solvent. Some components of the formulation may perform more than one function.

A compound for use in the invention may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, a compound for use in the invention may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

A compound for use in the invention may also be administered directly into the blood stream, into muscle, or into an internal organ. Such parenteral administration includes intravenous, intraarterial, intraperitoneal, intrathecal, intraventricular, intraurethral, intrasternal, intracranial, intramuscular and subcutaneous administration. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of a compound used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus a compound for use in the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

A compound for use in the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm. Sci., 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

A compound for use in the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of a compound for use in the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of a compound for use in the invention, a suitable powder base such as lactose or starch and a performance modifier such as /-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound for use in the invention per actuation and the actuation volume may vary from 1 μl to 100 μl.

A typical formulation may comprise a compound for use in the invention, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units are typically arranged to administer a metered dose or “puff”. The overall daily dose will be administered in a single dose or, more usually, as divided doses throughout the day.

A compound for use in the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

A compound for use in the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

A compound for use in the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO-A-91/11172, WO-A-94/02518 and WO-A-98/55148.

The two components of the present combination invention (i.e. the selective noradrenaline reuptake inhibitor (NRI) and the phosphodiesterase type 5 (PDEV) inhibitor) may be administered simultaneously, sequentially or separately in order to enjoy the benefits of the combination therapy provided by the present invention. Each component may be administered on its own but is more usually, administered in association with one or more excipients as one of the pharmaceutical compositions described above. Usually, both components will be administered via the same route (e.g. the oral route). However, there may be circumstances where it is preferable to administer each component via a different route (e.g. one component via the oral route and one component via the parenteral route). For simultaneous administration, the two components preferably form part of the same pharmaceutical composition and are therefore administered via the same route.

Oral administration is preferred for both components of the invention. Most preferably, the two components are delivered simultaneously via the oral route, for example in the form of a tablet.

The two components of the present combination invention may conveniently be combined in the form of a kit. Such a kit comprises a selective noradrenaline reuptake inhibitor (NRI) and a phosphodiesterase type 5 (PDEV) inhibitor, each usually in the form of one of the pharmaceutical compositions described above, and means for separately retaining them, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering separate compositions at different dosage intervals, or for titrating separate compositions against one another. To assist compliance, the kit typically comprises directions for administration and may be provided with a so-called memory aid.

Determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients renders impractical the use of this form of testing as a primary model for synergy. However, the observation of synergy in one species can be predictive of the effect in other species and animal models exist, as described herein, to measure a synergistic effect and the results of such studies can also be used to predict effective dose and plasma concentration ratio ranges and the absolute doses and plasma concentrations required in other species by the application of pharmacokinetic/pharmacodynamic methods. Established correlations between animal models and effects seen in man suggest that synergy in animals is best-demonstrated using static and dynamic allodynia measurements in rodents that have undergone surgical (e.g. chronic constriction injury) or chemical (e.g. streptozocin) procedures to induce the allodynia. Because of plateau effects in such models, their value is best assessed in terms of synergistic actions that in neuropathic pain patients would translate to dose-sparing advantages. Other models in which existing agents used for the treatment of neuropathic pain give only a partial response are more suited to predict the potential of combinations acting synergistically to produce increased maximal efficacy at maximally tolerated doses of the two components.

Thus, as a further aspect of the present invention, there is provided a synergistic combination for human administration comprising a selective noradrenaline reuptake inhibitor (NRI), for example (S,S)-reboxetine or a pharmaceutically acceptable salt or solvate thereof, particularly (S,S)-reboxetine succinate, and a PDEV inhibitor, or a pharmaceutically acceptable salt or solvate thereof, in a w/w combination range which corresponds to the absolute ranges observed in a non-human animal model, preferably a rat model, primarily used to identify a synergistic interaction. Suitably, the ratio range in humans corresponds to a non-human range selected from between 1:50 to 50:1 parts by weight, 1:50 to 20:1, 1:50 to 10:1, 1:50 to 1:1, 1:20 to 50:1, 1:20 to 20:1, 1:20 to 10:1, 1:20 to 1:1, 1:10 to 50:1, 1:10 to 20:1, 1:10 to 10:1, 1:10 to 1:1, 1:1 to 50:1, 1.1 to 20:1 and 1:1 to 10:1.

When the selective NRI is (S,S)-reboxetine and the PDEV inhibitor is sildenafil, the human range corresponds to a synergistic dose range in a non-human, preferably rat, model of the order of 20:1 to 1:10, preferably 10:1 to 1:5, more preferably 5:1 to 2:5.

When the selective NRI is (S,S)-reboxetine and the PDEV inhibitor is vardenafil, the human range corresponds to a synergistic dose range in a non-human, preferably rat, model of the order of 20:1 to 1:10, preferably 10:1 to 1:5, and more preferably 5:1 to 2:5.

When the selective NRI is (S,S)-reboxetine and the PDEV inhibitor is tadalafil, the human range corresponds to a synergistic dose range in a non-human, preferably rat, model of the order of 20:1 to 1:10, preferably 10:1 to 1:5, and more preferably 5:1 to 2:5.

When the selective NRI is (S,S)-reboxetine and the PDEV inhibitor is 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (Compound A), the human range corresponds to a synergistic dose range in a non-human, preferably rat, model of the order of 20:1 to 1:10, preferably 10:1 to 1:5, more preferably 5:1 to 2:5, and most preferably 4:1 to 1:2. In one particular example, the human range corresponds to a synergistic dose range in a non-human, preferably rat, model of 3:1. In another particular example, the human range corresponds to a synergistic dose range in a non-human, preferably rat, model of 1:1.

For humans, several experimental pain models may be used in man to demonstrate that agents with proven synergy in animals also have effects in man compatible with that synergy. Examples of human models that may be fit for this purpose include the heat/capsaicin model (Petersen, K. L. & Rowbotham, M. C. (1999) NeuroReport 10, 1511-1516), the i.d capsaicin model (Andersen, O. L., Felsby, S., Nicolaisen, L., Bjerring, P., Jsesn, T. S. & Arendt-Nielsen, L. (1996) Pain 66, 51-62), including the use of repeated capsaicin trauma (Witting, N., Svesson, P., Arendt-Nielsen, L. & Jensen, T. S. (2000) Somatosensory Motor Res. 17, 5-12), and summation or wind-up responses (Curatolo, M. et al. (2000) Anesthesiology 93, 1517-1530). With these models, subjective assessment of pain intensity or areas of hyperalgesia may be used as endpoints, or more objective endpoints, reliant on electrophysiological or imaging technologies (such as functional magnetic resonance imaging) may be employed (Bornhovd, K., Quante, M., Glauche, V., Bromm, B., Weiller, C. & Buchel, C. (2002) Brain 125, 1326-1336). All such models require evidence of objective validation before it can be concluded that they provide evidence in man of supporting the synergistic actions of a combination that have been observed in animal studies.

For the present invention in humans, a suitable NRI:PDEV inhibitor ratio range is selected from between 1:50 to 50:1 parts by weight, 1:50 to 20:1, 1:50 to 10:1, 1:50 to 1:1, 1:20 to 50:1, 1:20, to 20:1, 1:20 to 10:1, 1:20 to 1:1, 1:10 to 50:1, 1:10 to 20:1, 1:10 to 10:1, 1:10 to 1:1, 1:1 to 50:1, 1.1 to 20:1 and 1:1 to 10:1, more suitably 1:10 to 20:1, preferably, 1:1 to 10:1.

Optimal doses of each component for synergy can be determined according to published procedures in animal models. However, in man (even in experimental models of pain) the cost can be very high for studies to determine the entire exposure-response relationship at all therapeutically relevant doses of each component of a combination. It may be necessary, at least initially, to estimate whether effects can be observed that are consistent with synergy at doses that have been extrapolated from those that give optimal synergy in animals. In scaling the doses from animals to man, factors such as relative body weight/body surface area, relative absorption, distribution, metabolism and excretion of each component and relative plasma protein binding need to be considered and, for these reasons, the optimal dose ratio predicted for man (and also for patients) is unlikely to be the same as the dose ratio shown to be optimal in animals. However, the relationship between the two can be understood and calculated by one skilled in the art of animal and human pharmacokinetics. Important in establishing the bridge between animal and human effects are the plasma concentrations obtained for each component used in the animal studies, as these are related to the plasma concentration of each component that would be expected to provide efficacy in man. Pharmacokinetic/pharmacodynamic modelling (including methods such as isobolograms, interaction index and response surface modelling) and simulations may help to predict synergistic dose ratios in man, particularly where either or both of these components has already been studied in man.

Thus, according to a further aspect of the present invention, there is provided a synergistic combination for administration to humans comprising a selective noradrenaline reuptake inhibitor (NRI), for example (S,S)-reboxetine or a pharmaceutically acceptable salt or solvate thereof, particularly (S,S)-reboxetine succinate, and a PDEV inhibitor, or pharmaceutically acceptable salts or solvates thereof, where the dose range of each component corresponds to the absolute synergistic ranges observed in a non-human animal model, preferably the rat model, primarily used to identify a synergistic interaction. Suitably, the dose range of selective noradrenaline reuptake inhibitor in human corresponds to a dose range of 0.5-50 mg/kg, more suitably 1-30 mg/kg, in the rat and the corresponding dose range for a PDEV inhibitor is 0.1-10 mg/kg, more suitably 1-10 mg/kg.

For (S,S)-reboxetine and sildenafil, the dose range in the human suitably corresponds to a synergistic range of 1-30 mg/kg. (S,S)-reboxetine and 0.5-10 mg/kg sildenafil in the rat. For (S,S)-reboxetine and vardenafil, the dose range in the human suitably corresponds to a synergistic range of 1-30 mg/kg (S,S)-reboxetine and 0.5-10 mg/kg vardenafil in the rat. For (S,S)-reboxetine and tadalafil, the dose range in the human suitably corresponds to a synergistic range of 1-30 mg/kg (S,S)-reboxetine and 0.5-10 mg/kg tadalafil in the rat. For (S,S)-reboxetine and 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (Compound A), the dose range in the human suitably corresponds to a synergistic range of 1-30 mg/kg (S,S)-reboxetine and 0.5-10 mg/kg Compound A in the rat.

For administration to human patients, the optimal total daily dose of the selective noradrenaline reuptake inhibitor (NRI) and the phosphodiesterase type 5 (PDEV) inhibitor administered according to the present invention will vary considerably according to the particular compounds chosen. Such optimal doses are readily determined by the skilled person in accordance with normal pharmaceutical practice. Suitably, the dose of a selective noradrenaline reuptake inhibitor (NRI) for use in a human is in a range selected from 0.05-1000 mg, 0.1-500 mg, 0.2-100 mg, 0.5-50 mg, 1-25 mg, 1 to 4 times per day, suitably once or twice a day, and the dose of PDEV inhibitor is in a range selected from 1-200 mg, 1-100 mg, 1-50 mg, 1-25 mg, 10-100 mg, 10-50 mg or 10-25 mg, suitably 10-100 mg, once, twice or three times per day, suitably once per day. As an example, in the case where the chosen selective noradrenaline reuptake inhibitor is (S,S)-reboxetine, the total daily dose is typically in the range 0.1 mg to 10 mg depending, of course, on the mode of administration. Preferably, the daily dose of (S,S)-reboxetine will be in the range 0.1 mg to 8 mg, most preferably in the range 0.5 mg to 6 mg. As a further example, in the case where the chosen PDEV inhibitor is sildenafil, a dose of from 10 to 100 mg (e.g. 25, 50 or 100 mg) is typically administered once, twice or three times a day, preferably once a day. As a further example, in the case where the chosen PDEV inhibitor is vardenafil, a dose of from 1 to 50 mg (e.g. 2.5, 5, 10 or 20 mg) is typically administered once or twice a day, preferably once a day. As a further example, in the case where the chosen PDEV inhibitor is tadalafil, a dose of from 1 to 50 mg (e.g. 5, 10 or 20 mg) is typically administered once, twice or three times a day, preferably once a day.

The total daily dose of either component may be administered in single or divided doses and may, at the physician's discretion, fall outside of the typical ranges described above. Such typical ranges are based on an average human subject having a weight of about 60 kg to 70 kg. The physician will readily be able to determine doses for subjects whose weight falls outside this range, such as infants and the elderly. Preferably, the two components of the present combination invention will be administered once or twice a day.

For the avoidance of doubt, references herein to “treatment” include references to curative, palliative and prophylactic treatment.

The combination of the present invention includes a selective noradrenaline reuptake inhibitor (NRI) and a phosphodiesterase type 5 (PDEV) inhibitor. The PDEV activity of the compounds used in the combination of the present invention can be determined by the following test methods.

In vitro PDE inhibitory activities against cyclic guanosine 3′,5′-monophosphate (cGM P) and cyclic adenosine 3′,5′-monophosphate (cAMP) phosphodiesterases were determined by measurement of their IC₅₀ values (the concentration of compound required for 50% inhibition of enzyme activity).

The required PDE enzymes were isolated from a variety of sources, including human corpus cavernosum, human and rabbit platelets, human cardiac ventricle, human skeletal muscle and bovine retina, essentially by the method of W. J. Thompson and M. M. Appleman (Biochem., 1971, 10, 311). In particular, the cGMP-specific PDE (PDE5) and the cGMP-inhibited cAMP PDE (PDE3) were obtained from human corpus cavernosum tissue, human platelets or rabbit platelets; the cGMP-stimulated PDE (PDE2) was obtained from human corpus cavernosum; the calcium/calmodulin (Ca/CAM)-dependent PDE (PDE1) from human cardiac ventricle; the cAMP-specific PDE (PDE4) from human skeletal muscle; and the photoreceptor PDE (PDE6) from bovine retina. Phosphodiesterases 7-11 were generated from full length human recombinant clones transfected into SF9 cells.

Assays were performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [³H]-labeled at a conc˜⅓ Kₘ) such that IC₅₀≅Kᵢ. The final assay volume was made up to 100 μl with assay buffer[20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension.

### Functional Activity

This was assessed in vitro by determining the capacity of a compound of the invention to enhance sodium nitroprusside-induced relaxation of pre-contracted rabbit corpus cavernosum tissue strips, as described by S. A. Ballard et al. (Brit. J. Pharmacol., 1996, 118 (suppl.), abstract 153P).

### In Vivo Activity

Compounds were screened in anaesthetised dogs to determine their capacity, after i.v. administration, to enhance the pressure rises in the corpora cavernosa of the penis induced by intracavernosal injection of sodium nitroprusside, using a method based on that described by Trigo-Rocha et al. (Neurourol. and Urodyn., 1994, 13, 71).

The combination of the present invention displays synergy in animal models of pain, as described in the following paragraphs.

### Methods

### Animals

Male Sprague Dawley rats (175-200 g at time of surgery), obtained from Charles River, Margate, U.K.) were housed in groups of 3-6. All animals were kept under a 12 h light/dark cycle (lights on at 07 h 00 min) with food and water ad libitum. All experiments were carried out by an observer blind to drug treatments.

### Chronic Constriction Injury (CCI) Model

Animals were placed in an anaesthetic chamber and anaesthetised with a 2% isofluorane O₂ mixture. The right hind thigh is shaved and swabbed with 1% iodine. Animals were then transferred to a homeothermic blanket for the duration of the procedure and anaesthesia maintained during surgery via a nose cone. The skin was cut along the line of the thigh bone. The common sciatic nerve was exposed at the middle of the thigh by blunt dissection through biceps femoris. Proximal to the sciatic trifurcation, about 7 mm of nerve was freed by inserting forceps under the nerve and the nerve gently lifted out of the thigh. The forceps were gently opened and closed several times to aid clearance of the fascia from the nerve. Suture was pulled under the nerve using forceps and tied in a simple knot until slight resistance was felt and then double knotted. The procedure was repeated until 4 ligatures (4-0 silk) were tied loosely around the nerve with approx 1 mm spacing. The incision was closed in layers and the wound treated with topical antibiotics.

### Assessment of Pain-Like Threshold

Animals were habituated to test cages prior to the assessment of allodynia. Static allodynia was evaluated by application of von Frey hairs (Stoelting, Wood Dale, Ill., USA) in ascending order of force (0.7, 1.2, 1.4, 2, 4, 6, 8, 10, 15 and 26 grams) to the plantar surface of hind paws. Each von Frey hair was applied to the paw for a maximum of 6 seconds, or until a withdrawal response occurred. Once a withdrawal response to a von Frey hair was established, the paw was re-tested, starting with the filament below the one that produced a withdrawal, and subsequently with the remaining filaments in descending force sequence until no withdrawal occurred. The highest force of 26 g lifted the paw as well as eliciting a response, thus represented the cut off point. Each animal had both hind paws tested in this manner. The lowest amount of force required to elicit a response was recorded as paw withdrawal threshold (PWT) in grams. Static allodynia was defined as present if animals responded to a stimulus of, or less than, 4 g, which is innocuous in normal rats.

### Compounds

(S,S)-reboxetine succinate was dissolved in Millipore filtered water and administered intraperitoneally (IP) at 1, 3, 10 and 30 mg/kg of the free base. This corresponds to 1.38, 4.13, 13.8 and 41.3 mg/kg of (S,S)-reboxetine succinate.

The PDEV inhibitor, 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (hereinafter referred to as Compound A) was dissolved in polyethylene glycol 400 and administered subcutaneously (SC) at 3, 10 and 30 mg/kg.

### Data Analysis

Static allodynia was graphically represented as median [LQ;UQ] in a logarithmic scale and data analysed by Kruskall-Wallis test for non-parametric results, followed by Mann-Whitney's U test vs vehicle group for each time point, using Prism Software version 3 (GraphPad, San Diego, USA).

### Results

The pharmacological effect of combined doses of (S,S)-reboxetine and Compound A was compared to the per se activity of the single compounds in the CCI rat model of neuropathic pain in order to establish a synergistic effect of the two medicaments. Normally, naïve rats, when tested for static allodynia with von Frey filaments, display a pain threshold to mechanical stimulus between 8 to 15 g. After CCI of sciatic rats develop chronic pain, which reflect in a significant decrease of the paw withdrawal threshold (PWT) to 2 g[0;0]. When treated with vehicle CCI rats do not show changes in the pain threshold but maintain a consistent value throughout the experiment.

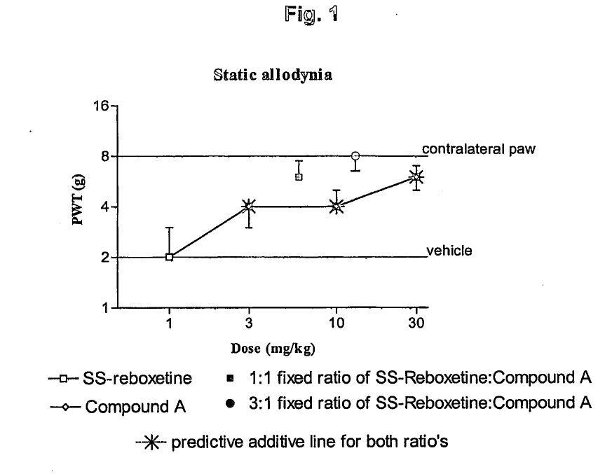

FIG. 1 shows the effects of S,S-reboxetine (1-3-10-30 mg/kg, IP), Compound A (3-10-30 mg/kg, SC) and the 1:1 and 3:1 ratio combination (3 or 10 mg/kg, IP, (S,S)-reboxetine and 3 mg/kg, SC, Compound A) of the two compounds in static allodynia in the CCI rat model. Data are the peak effects (1 h post administration) for each compound and doses expressed as median [LQ; UQ] of 6 rats per group. The dotted line shows the predicted additive line as previously described by Tallarida, “Drug synergism: Its detection and application”; JPET (2001), 298, 865-872. In this specific case it corresponds to the Compound A dose-response line.

Following single administration both (S,S)-reboxetine and Compound A produced a dose dependent anti-allodynic effect in the preclinical model of chronic pain. In particular the highest dose of both compounds (30 mg/kg) produced a reversal (6 g[1,0] and 6 g[0;1] for (S,S)-reboxetine and PDEV, respectively) compared to the vehicle treated group.

The effect of a combination of (S,S)-reboxetine and a PDEV inhibitor was explored at the 1:1 and 3:1 ratio using 3 or 10 mg/kg of (S,S)-reboxetine and 3 mg/kg of Compound A. As shown in FIG. 1, both combinations of the two compounds produced an anti-allodynic effect which results in a superior effect than seen with either compound when used alone or the predictive additive effect (indicated by the dotted line). This suggests a synergistic interaction in the pharmacology of the two compounds.

Some further advantages of the combination provided by the present invention may be appreciated using the pain models described below.

### Streptozocin-Induced Neuropathy

Animals are administered streptozocin (50 mg/kg i.p.) to induce diabetes. Two weeks later animals are assessed for the onset of pain symptoms and all experiments are carried out after this time.

### MIA-Induced Osteoarthritis

Animals are placed in an anaesthetic chamber and anaesthetised with a 2% isofluorane/oxygen mixture. A single injection of monosodium iodoacetate (2 mg/25 μl) is administered intraarticularly through the patella ligament. Anaesthesia is maintained via a nose cone for the length of the injection.

### Carrageenan-Induced Thermal Hyperalgesia

Thermal hyperalgesia is assessed using the rat plantar test (Ugo Basile, Italy) following a modified method of Hargreaves et al., 1988. Rats are habituated to an apparatus consisting of three individual perspex boxes on an elevated glass table. A mobile radiant heat source is located under the table and focused onto the hind paw and paw withdrawal latencies are recorded. There is an automatic cut off point of 22.5 seconds to prevent tissue damage. Paw withdrawal latencies are taken two to three times for both hind paws of each animal, the mean of which represents baselines for right and left hind paws. The apparatus is calibrated to give a paw withdrawal latency of approximately 10 seconds.

In these pain models, the further end-points described below are used.

### Dynamic Allodynia

Dynamic allodynia is assessed by lightly stroking the plantar surface of the hind paw with a cotton bud. Care is taken to perform this procedure in fully habituated rats that are not active to avoid recording general motor activity. At least three measurements are taken at each time point the mean of which represented the paw withdrawal latency. If no reaction is exhibited within 15 seconds the procedure is terminated and animals are assigned this withdrawal time. Thus 15 seconds effectively represents no withdrawal. A withdrawal response is often accompanied with repeated flinching or licking of the paw. Dynamic allodynia is considered to be present if animals respond to the cotton stimulus before 8 seconds of stroking.

Dose-responses are first performed for each component of the combination alone. A combination is then examined following a fixed ratio design. A dose-response to each fixed dose ratio of the combination is performed. On each test day, baseline paw withdrawal thresholds to von Frey hairs and paw withdrawal latencies to a cotton bud stimulus are determined prior to drug treatment. After drug administration, paw withdrawal thresholds and paw withdrawal latencies are re-examined for up to 5 hours. The data are expressed at the two hour time point for both the static and dynamic data as this time point represents the peak anti-allodynic effect.

### Weight Bearing

Animals are examined for hypersensitivity in the weight-bearing test, using an “incapacitance tester” (Linton Instruments, Diss, Norfolk, U.K.). Rats are positioned with their fore limbs up on a perspex slope and hind limb weight distribution is measured via force transducers under each of the hind paws. Each animal is placed in the apparatus and the weight load exerted by the hind paws is noted. The difference in weight bearing is calculated by subtracting the ipsilateral (nerve injured) paw from the contralateral (normal) paw and this constitutes the raw data.

A combination of the present invention may be further combined with another pharmacologically active compound, or with two or more other pharmacologically active compounds, particularly in the treatment of pain. Thus, a combination of the present invention, in its broadest sense or in any of the preferred aspects presented above, may be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4 -(phosphonomethyl)-2-piperidinecarboxylic acid, budipine, EN-3231 (MorphiDex®), a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/ID} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-buten-1-amine (RJR-2403), (R)-5-(2-azetidinylmethoxy)-2-chloropyridine (ABT-594) or nicotine;
Tramadol®;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1 R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3 pyridinecarbonitrile, 2-[[(1 R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1 S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl),1,4-benzoquinone (CV-6504);
a sodium channel blocker, such as lidocaine;
a 5-HT3 antagonist, such as ondansetron;
   and the pharmaceutically acceptable salts and solvates thereof.

1. A combination consisting of:
(a) (S,S)-reboxetine or a pharmaceutically acceptable salt or solvate thereof; and
(b) a phosphodiesterase type 5 (PDEV) inhibitor.

2. A combination as claimed in claim 1 wherein the phosphodiesterase type 5 (PDEV) inhibitor is selected from:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) or a pharmaceutically acceptable salt or solvate thereof;
(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (tadalafil, IC-351, Cialis®) or a pharmaceutically acceptable salt or solvate thereof;
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) or a pharmaceutically acceptable salt or solvate thereof;
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one or a pharmaceutically acceptable salt or solvate thereof;
5-(5-acetyl-2-butoxy-3-pyridinyl)-3-ethyl-2-(1-ethyl-3-azetidinyl)-2,6-dihydro-7H-pyrazolo[4,3-d]pyrimidin-7-one or a pharmaceutically acceptable salt or solvate thereof;
1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}-4-ethylpiperazine or a pharmaceutically acceptable salt or solvate thereof;
N-[1-(2-ethoxyethyl)-5-(N-ethyl-N-methylamino)-7-(4-methylpyridin-2-ylamino)-1H-pyrazolo[4,3-d]pyrimidine-3-carbonyl]methanesulfonamide or a pharmaceutically acceptable salt or solvate thereof;
3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one or a pharmaceutically acceptable salt or solvate thereof; and
{3-ethyl-5-[(R)-3-methyl-piperazin-1-yl]-1-[2-(2,2,2-trifluoroethoxy)-ethyl]-1H-pyrazolo[4,3-d]pyrimidin-7-yl}-pyrimidin-4-yl-amine or a pharmaceutically acceptable salt or solvate thereof.

3. A pharmaceutical composition consisting of:
(a) (S,S)-reboxetine or a pharmaceutically acceptable salt or solvate thereof;
(b) a phosphodiesterase type 5 (PDEV) inhibitor; and
(c) a pharmaceutically acceptable excipient, diluent or carrier.

4. A combination according to claim 1 or 2 for use as a medicament.

5. Use of a combination according to claim 1 or 2 in the manufacture of a medicament for simultaneous, sequential or separate administration of both agents in the treatment of pain.

6. A method of treating pain comprising administering simultaneously, sequentially or separately, to a mammal in need of such treatment, an effective amount of a combination according to claim 1 or 2.

7. Use of a combination according to claim 1 or 2 in the manufacture of a medicament for simultaneous, sequential or separate administration of both agents in the treatment of incontinence.

8. Use according to claim 7, wherein the incontinence is selected from stress incontinence, genuine stress incontinence, urge incontinence and mixed incontinence.

9. A method of treating incontinence comprising administering simultaneously, sequentially or separately, to a mammal in need of such treatment, an effective amount of a combination according to claim 1 or 2.

10. A method according to claim 9, wherein the incontinence is selected from stress incontinence, genuine stress incontinence, urge incontinence and mixed incontinence.

11. A kit consisting essentially of:
(a) (S,S)-reboxetine or a pharmaceutically acceptable salt or solvate thereof;
(b) a phosphodiesterase type 5 (PDEV) inhibitor; and
(c) means for containing said compounds.
